(19) United States
(12) Patent Application Publication
(10) Pub. No.: US 2022/0298522 A1
(43) Pub. Date: Sep. 22, 2022

(54) METHODS OF CONTROLLING CANNABINOID SYNTHESIS IN PLANTS OR CELLS AND PLANTS AND CELLS PRODUCED THEREBY

(71) Applicant: The State of Israel, Ministry of Agriculture & Rural Development, Agricultural Research Organization, Rishon-LeZion (IL)

(72) Inventors: Moshe Arie FLAISHMAN, Herzliya (IL); Hadas SHAFRAN-TOMER, Rehovot (IL); Reut COHEN PEER, Kiryat-Ono (IL); Oded COHEN, Nir Zvi (IL)

(73) Assignee: The State of Israel, Ministry of Agriculture & Rural Development, Agricultural Research Organization, Rishon-LeZion (IL)

(21) Appl. No.: 17/631,510

(22) PCT Filed: Jul. 29, 2020
(86) PCT No.: PCT/IL2020/050835

§ 371 Date: Jan. 31, 2022

Related U.S. Application Data
(60) Provisional application No. 62/880,136, filed on Jul. 30, 2019.

(51) Int. Cl. C12N 15/82; C07K 14/415

(52) U.S. Cl. CPC C12N 15/8243; C07K 14/415

(57) ABSTRACT

A method of controlling cannabinoid synthesis in a cell or plant or plant part comprising same is provided. The method comprising modulating expression in the cell of at least one polypeptide comprising an amino acid sequence at least 95% identical to SEQ ID NO: 1-15 and 18-86, the polypeptide modulating cannabinoid synthesis, thereby controlling cannabinoid synthesis in the cell. Also provided are methods of producing cannabinoids and selecting plants producing cannabinoids of interest.

### RELATED APPLICATION/S

This application claims the benefit of priority from U.S. Provisional Patent Application No. 62/880,136 filed Jul. 30, 2019 which is hereby incorporated by reference.

### SEQUENCE LISTING STATEMENT

The ASCII file, entitled 83866 SequenceListing.txt, created on 28 Jul. 2020, comprising 700,416 bytes, submitted concurrently with the filing of this application is incorporated herein by reference.

### FIELD AND BACKGROUND OF THE INVENTION

The present invention, in some embodiments thereof, relates to methods of controlling cannabinoid synthesis in plants or cells and plants and cells produced thereby.

Cannabis sativa is an annual flowering plant from Cannabaceae family. It is also known by other names, such as cannabis, marijuana, ganja and hemp. This plant has been used for industrial, medicinal and recreational. The plant Cannabis sativa contains a number of chemical compounds termed cannabinoids, which are known by their pharmaceutical potential. Recently, the usage of Cannabis for medicinal purposes has been legalized in many countries (Volkow, et al., 2017).

Recent findings suggest that different phytocannabinoids exhibit diverse pharmacological and biological activities, acting on multiple targets. Russo (2011) supports this assumption by stating that phytocannabinoids and combinations of cannabinoids can, in certain situations, be more effective than Δ9-THC or CBD alone.

Thus, today's research is focused on different cannabinoids in combination with other Cannabis-derived compounds and their effect on the treatment of various diseases. Cannabis's value and potential is changing all over the world. Patients, physicians, and governmental bodies are giving increased attention to medical Cannabis. In the past ten years, there has been a rapid growth in the discovery and use of Cannabis-based extracts for various therapeutic and medical purposes. The number of people worldwide that are currently using physician-prescribed medical Cannabis is estimated at millions. According to the ProCon organization, in the U.S. alone, as of 2018, this number was over 2.1 million patients.

Phytocannabinoids are terpenophenolic compounds associated with the effects of the Cannabis plant and mimic the effects of endogenous cannabinoids. These phytocannabinoids are biosynthesized and secreted by glandular trichomes found on the flower tops of the Cannabis plant. In the 1960s several cannabinoids were discovered, including cannabigerol (CBG), tetracannabivarin (THCV), and cannabichromene (CBC). Currently 144 have been isolated. C. sativa contains phytocannabinoids, chemical compounds that can be classified into 11 types: cannabidiol (CBD), cannabinol (CBN), cannabinodiol (CBDN), cannabichromene (CBC), cannabigerol (CBG), cannabicyclol (CBL), cannabielsoin (CBE), cannabitriol (CBT), A9 tetrahydrocannabinol (Δ9-THC), and Δ8-tetrahydrocannabinol (Δ8-THC) and miscellaneous types (Hanuš, et al., 2016). Phytocannabinoids are biosynthesized as acids. In general, CBG, Δ9-THC, CBD and CBC phytocannabinoid subclasses are biosynthesized in Cannabis plants, while the remaining six subclasses are probably the result of decomposition either in the plant or due to poor storage conditions following harvest. All subclasses of phytocannabinoids derive initially from CBG-type ones, and therefore bear similarity in terms of chemical. trans-Δ9-tetrahydrocannabinolic acid (Δ9-THCA), cannabidiolic acid (CBDA), and cannabichromenic acid (CBCA) differ only by the enzymatic cyclization of the terpene moiety (Kinghorn, et al., 2017).

Cannabis strains significantly vary in their chemical compositions. The concentration of Cannabis's compounds depends on the plant's tissue-type, age, variety, growth conditions (nutrition, humidity and light levels), harvest time, and storage conditions. Generally, marijuana has high amount of Δ9-tetrahydrocannabinol (Δ9-THC) and low amount of cannabidiol (CBD). Hemp or industrial hemp, contains high amount of CBD and very low in Δ9-THC. Analyzing the chemical content of the plants is of major importance considering that the concentrations of these constituents and their interplay may determine medicinal effects and adverse side effects. Major and minor phytocannabinoids can have remarkably positive effects in mammalian behavior related to anxiety and drug acquisition and may offer novel drug abuse treatment options. The ratios of these major and minor compounds can vary greatly and some compounds are not often detected or tested for or reported. Turner et al. (2017) and Morales et al. (2017), suggested that the relative proportions of each phytocannabinoid type will additionally influence the pharmacological effects of whole Cannabis extracts, either through a polypharmacological effect of the phytocannabinoids themselves, or through modulation of phytocannabinoid effects by the non-cannabinoid content of the plant since they act on multiple targets.

### SUMMARY OF THE INVENTION

According to an aspect of some embodiments of the present invention there is provided a method of controlling cannabinoid synthesis in a cell or plant or plant part comprising same, the method comprising modulating expression in the cell of at least one polypeptide comprising an amino acid sequence at least 95% identical to SEQ ID NO: 1-15 and 18-86, the polypeptide modulating cannabinoid synthesis, thereby controlling cannabinoid synthesis in the cell.

According to an aspect of some embodiments of the present invention there is provided a method of producing cannabinoids in a plant, the method comprising modulating expression in the plant of at least one polypeptide comprising an amino acid sequence at least 95% identical to SEQ ID NO: 1-15 and 18-86 the polypeptide modulating cannabinoid synthesis, thereby producing cannabinoids in the cell.

According to an aspect of some embodiments of the present invention there is provided a method of selecting a plant for a cannabinoid profile, the method comprising analyzing in the plant or part thereof presence of a nucleic acid sequence at least 95% identical to SEQ ID NO: 91-180 or amino acid sequence at least 95% identical to SEQ ID NO: 1-15 and 18-86, wherein presence or absence of the nucleic acid sequence or amino acid sequence is indicative of the cannabinoid profile.

According to some embodiments of the invention, the method further comprises determining a cannabinoid or cannabinoid profile of the plant or part thereof.

According to some embodiments of the invention, the method further comprises recovering the cannabinoids from the plant or cell.

According to some embodiments of the invention, the recovering is by extraction and/or fractionation.

According to an aspect of some embodiments of the present invention there is provided a nucleic acid construct comprising a nucleic acid sequence encoding a polypeptide at least 95% identical to SEQ ID NO: 1-15 and 18-86, the polypeptide modulating cannabinoid synthesis, and another nucleic acid sequence comprising a cis-acting regulatory region heterologous to the nucleic acid sequence and capable of regulating expression of the polypeptide.

According to an aspect of some embodiments of the present invention there is provided a cell, a plant, or part thereof having being genetically modified to express a polypeptide comprising an amino acid sequence at least 95% identical to SEQ ID NO: 1-15 and 18-86, the polypeptide modulating cannabinoid synthesis.

According to an aspect of some embodiments of the present invention there is provided a cell, a plant, or part thereof having being genetically modified to down-regulate expression of a polypeptide comprising an amino acid sequence at least 95% identical to SEQ ID NO: 1-15 and 18-86.

According to some embodiments of the invention, the cell, plant or part thereof of is a transgenic plant or plant cell.

According to some embodiments of the invention, the cell, plant or part thereof of claim 8 or 9 being a non-transgenic plant or plant cell.

According to some embodiments of the invention, the modulating is by genome editing.

According to some embodiments of the invention, the modulating is by transgenesis.

According to some embodiments of the invention, the modulating is by breeding.

According to some embodiments of the invention, the modulating comprises upregulating expression.

According to some embodiments of the invention, the modulating comprises downregulating expression.

According to some embodiments of the invention, the cell is yeast.

According to some embodiments of the invention, the method further comprises supplementing the cell with at least one cannabinoid or precursor thereof and/or enzyme modulating cannabinoid synthesis.

According to some embodiments of the invention, the cell is a plant cell.

According to some embodiments of the invention, the plant part is a flower.

According to some embodiments of the invention, the plant part is a seed.

According to some embodiments of the invention, the plant part is a root.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a diagram showing a phylogenetic analysis of the three Cannabis genomes comped to THCA synthase (THCAS), CBDA synthase (CBDAS), CBCA synthase (CBCAS) and CBGA synthase (CBGAS)-like genes. All 8 groups of newly discovered genes are depicted here.

FIG. 2 is a Table showing Gene expression profiles taken from cannabis PK plant tissue at different developmental stages: a heat map shows the relative expression values (log 2 RPKM) of the cannabinoids synthase candidate genes, in PK plant tissue.

FIG. 3 is a diagram showing a phylogenetic tree of CBCAS like genes (group I) according to some embodiments of the invention;

FIG. 4 is an illustration demonstrating elements common to promoters in group I (CBCAS like genes): CCAF (Circadian clock associated), DREB (a-biotic stress element), EINL (Ethylen insensitive 3 like factors), GAPB (GAP-Box (light response elements)), HEAT (Heat shock factors), IBOX (light regulation), STKM (Storekeeper motif), TOEF (Target of early activation tagged factors-AP2 domain).

FIG. 5 is a graphic display of the sequence similarity (DNA and Protein), in the group II (THCAS-like genes). Display by AlignX of vector NTi software.

FIG. 6 is an illustration demonstrating elements common to promoters in the group: CCAF (Circadian clock associated), HEAT (Heat shock factors), IBOX (light regulation).

FIG. 7 is a diagram showing a phylogenetic tree of group 3. The phylogenetic tree assembly using Vector NTi, AlignX default.

FIGS. 8A-B show promoter analysis demonstrating all element common to all sequences in the third group. FIG. 8A—upper branches showing CCAF (Circadian clock associated), HEAT (Heat shock factors), IBOX (light regulation). FIG. 8B—the lower group, show CCAF (Circadian clock associated), EINL (Ethylen insensitive 3 like factors), HEAT (Heat shock factors), IBOX (light regulation), LREM (Light responsive element motif), and TOEF (Target of early activation tagged factors-AP2 domain).

FIG. 9 is a diagram showing phylogenetic analysis of group 4 (CBDAS like genes).

FIG. 10 shows promoter analysis demonstrating elements common to all sequences in group 4: CCAF (Circadian clock associated), DREB (a-biotic stress element), HEAT (Heat shock factors), IBOX (light regulation).

FIG. 11 is a diagram showing phylogenetic analysis of group 5 (CBGAS like genes).

FIG. 12 is an illustration showing promoter analysis demonstrating elements common to all sequences in group 5: CCAF (Circadian clock associated), GAPB (GAP-Box (light response elements), HEAT (Heat shock factors), IBOX (light regulation), LREM, TOEF (Target of early activation tagged factors-AP2 domain).

FIG. 13 is an illustration showing promoter analysis demonstrating elements in group 6: CCAF (Circadian clock associated), CE1F, DREB (a-biotic stress element), EINL (Ethylen insensitive 3 like factors), GAPB (GAP-Box (light response elements)), HEAT (Heat shock factors), IBOX (light regulation), LREM, TOEF (Target of early activation tagged factors-AP2 domain).

FIG. 14 is a diagram showing phylogenetic analysis of group 7.

FIG. 15 is an illustration of promoter analysis demonstrating elements common to all sequences in group 7: CCAF (Circadian clock associated), GAPB (GAP-Box (light response elements)), HEAT (Heat shock factors), IBOX (light regulation), STKM (Storekeeper motif).

FIG. 16 is a diagram showing phylogenetic analysis of group 8.

FIG. 17 is an illustration of promoter analysis demonstrating all element common to all sequences in group 8: GAPB (GAP-Box (light response elements)), HEAT (Heat shock factors) and TOEF (Target of early activation tagged factors-AP2 domain).

FIGS. 18A-B are schemes of pK7WG2 plasmid constructs for over expressed THC synthase (FIG. 18A) and CBD synthase (FIG. 18B) genes.

FIGS. 19A-E are images of Agrobacterium mediated transformation in callus cultures of C. sativa (#201). Leaf explants were collected from the proliferated shoots of C. sativa (FIG. 19A), which formed a callus after 1 week of incubation on CRF medium (FIG. 19B); with a substantial callus growth after 1 month (FIG. 19C) that showed GUS positive results after 3 days (FIG. 19D) as transient and 10 days (FIG. 19E).

FIGS. 20A-B are images showing GUS overexpression (FIG. 20A) and PCR analysis (FIG. 20B) of callus cultures of C. sativa (#201) 30 days after transformation.

FIGS. 21A-B are graphs showing over expression of THCAS (FIG. 21A) or CBDAS (FIG. 21B) in callus cultures of C. sativa. W.T.=WILD TYPE; O.E.=Over expression transgenic callus.

### DESCRIPTION OF SPECIFIC EMBODIMENTS OF THE INVENTION

The present invention, in some embodiments thereof, relates to methods of controlling cannabinoid synthesis in plants or cells and plants and cells produced thereby.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In order to identify genes associated with cannabinoid synthesis, the present inventors combined DNA sequencing and expression data analysis. The present inventors applied bioinformatics tools to in silico identify genes showing homology to the phytocannabinoid subclasses CBG, Δ9-THC, CBD and CBC phytocannabinoid. Gene expression profiling of the newly identified genes was performed in cannabis plant tissues at different developmental stages. In addition the DNA promoter region that initiates transcription of each gene was identified and the type of binding sites found in the DNA of each gene was characterized.

Hence the identification of novel genes associated with phytocannabinoid synthesis can be used in regulating the phytocannabionoid profile in plants and in selection of such plants.

Thus, according to an aspect of the invention there is provided a method of controlling cannabinoid synthesis in a cell or plant or plant part comprising same, the method comprising modulating expression in the cell of at least one polypeptide comprising an amino acid sequence at least 95% identical to SEQ ID NO: 1-15 and 18-86, the polypeptide modulating cannabinoid synthesis, thereby controlling cannabinoid synthesis in the cell.

According to an additional or alternative aspect there is provided a method of producing cannabinoids in a plant, the method comprising modulating expression in the plant of at least one polypeptide comprising an amino acid sequence at least 95% identical to SEQ ID NO: 1-15 and 18-86 the polypeptide modulating cannabinoid synthesis, thereby producing cannabinoids in the cell.

As used herein “controlling” refers to artificially (man-made activity) interfering with the natural process of cannabinoid synthesis in the cell and shifting it to a profile of interest. The term can be interchanged with “regulating” or “modulating” or “governing” or “orchestrating”.

As used herein, a “cannabinoid” is a chemical compound (such as cannabinol, THC or cannabidiol) that is found in the plant species Cannabis among others like Echinacea; Acmella Oleracea; Helichrysum Umbraculigerum; Radula Marginata (Liverwort) and Theobroma Cacao, and metabolites and synthetic analogues thereof that may or may not have psychoactive properties. Cannabinoids therefore include (without limitation) compounds (such as THC) that have high affinity for the cannabinoid receptor (for example Ki<250 nM), and compounds that do not have significant affinity for the cannabinoid receptor (such as cannabidiol, CBD). Cannabinoids also include compounds that have a characteristic dibenzopyran ring structure (of the type seen in THC) and cannabinoids which do not possess a pyran ring (such as cannabidiol). Hence a partial list of cannabinoids includes THC, CBD, dimethyl heptylpentyl cannabidiol (DMHP-CBD), 6,12-dihydro-6-hydroxy-cannabidiol (described in U.S. Pat. No. 5,227,537, incorporated by reference); (3 S,4R)-7-hydroxy-.DELTA.6-tetrahydrocannabinol homologs and derivatives described in U.S. Pat. No. 4,876,276, incorporated by reference; (+)-4-[4-DMH-2,6-diacetoxy-phenyl]-2-carboxy-6,6-dimethylbicyclo[3.1.1]he-pt-2-en, and other 4-phenylpinene derivatives disclosed in U.S. Pat. No. 5,434,295, which is incorporated by reference; and cannabidiol (−)(CBD) analogs such as (−)CBD-monomethylether, (−)CBD dimethyl ether; (−)CBD diacetate; (−)3′-acetyl-CBD monoacetate; and .+−.AF11, all of which are disclosed in Consroe et al., J. Clin. Pharmacol. 21:428S-436S, 1981, which is also incorporated by reference. Many other cannabinoids are similarly disclosed in Agurell et al., Pharmacol. Rev. 38:31-43, 1986, which is also incorporated by reference.

Examples of cannabinoids are tetrahydrocannabinol, cannabidiol, cannabigerol, cannabichromene, cannabicyclol, cannabivarin, cannabielsoin, cannabicitran, cannabigerolic acid, cannabigerolic acid monomethylether, cannabigerol monomethylether, cannabigerovarinic acid, cannabigerovarin, cannabichromenic acid, cannabichromevarinic acid, cannabichromevarin, cannabidolic acid, cannabidiol monomethylether, cannabidiol-C4, cannabidivarinic acid, cannabidiorcol, delta-9-tetrahydrocannabinolic acid A, delta-9-tetrahydrocannabinolic acid B, delta-9-tetrahydrocannabinolic acid-C4, delta-9-tetrahydrocannabivarinic acid,delta-9-tetrahydrocannabivarin, delta-9-tetrahydrocannabiorcolic acid, delta-9-tetrahydrocannabiorcol,delta-7-cis-iso-tetrahydrocannabivarin, delta-8-tetrahydrocannabiniolic acid, delta-8-tetrahydrocannabinol, cannabicyclolic acid, cannabicylovarin, cannabielsoic acid A, cannabielsoic acid B, cannabinolic acid, cannabinol methylether, cannabinol-C4, cannabinol-C2, cannabiorcol, 10-ethoxy-9-hydroxy-delta-6a-tetrahydrocannabinol, 8,9-dihydroxy-delta-6a-tetrahydrocannabinol, cannabitriolvarin, ethoxy-cannabitriolvarin, dehydrocannabifuran, cannabifuran, cannabichromanon, cannabicitran, 10-oxo-delta-6a-tetrahydrocannabinol, delta-9-cis-tetrahydrocannabinol, 3,4,5,6-tetrahydro-7-hydroxy-alpha-alpha-2-trimethyl-9-n-propyl-2,6-metha-no-2H-1-benzoxocin-5-methanol-cannabiripsol, trihydroxy-delta-9-tetrahydrocannabinol, and cannabinol.

As mentioned, other plants are also contemplated, especially those which are equipped with a cannabionoid synthesis mechanism. These include, but are not limited to, Phytocannabinoids are known to occur in several plant species besides cannabis. These include Echinacea purpurea, Echinacea angustifolia, Acmella oleracea, Helichrysum umbraculigerum, Humulus lupulus and Radula marginata.

In an additional embodiment, also contemplated are plant cells or even-non-plant cells e.g., yeast, which are devoid of a cannabinoid synthesis mechanism. These can be modified or supplemented with the relevant enzymes including those contemplated herein and substances to arrive at a functional cannabinoid producing plant, plant cell or another type of cell altogether e.g., yeast.

Thus embodiments of the invention contemplate genetically engineering “non-cannabinoid or cannabinoid analog producing cells” with a nucleic acid sequence as contemplated herein (involved in the production of cannabinoids). Non-cannabinoid or cannabinoid analog producing cells refer to a cell from any organism that does not produce a cannabinoid or cannabinoid analog. Illustrative cells include but are not limited to plant cells, as well as insect, mammalian, yeast, fungal, algal, or bacterial cells.

“Fungal cell” refers to any fungal cell that can be transformed with a gene encoding a cannabinoid or cannabinoid analog biosynthesis enzyme and is capable of expressing in recoverable amounts the enzyme or its products. Illustrative fungal cells include yeast cells such as Saccharomyces cerevisiae and Pichia pastoris. Cells of filamentous fungi such as Aspergillus and Trichoderma may also be used.

According to a specific embodiment, such a cell is a yeast cell.

Cannabinoid synthesis in yeast can be done using methods known in the art. For example, Laverty et al. Described expression in P. pastoris strains. Following is a non-limiting embodiment.

CBCAS can be amplified from DNA isolated from FN leaves using gene-specific primers PCR products and cloned into pPICz-alpa B. The expression vectors are then transformed into P. pastoris strain X-33 (Invitrogen) by electroporation. Positive recombinants can be selected for by plating transformed cells on YPD plates supplemented with 25 μg/mL phleomycin. To screen for activity, colonies are used to inoculate 5 mL BMG cultures, which can grow for 2 d at 37° C. with shaking. The cells are then pelleted by centrifugation, and grown for 4 d at 20° C. with shaking with the addition of 1% methanol daily. Enzyme activity can be tested by directly adding CBGA to clarified culture media, incubating and then analyzing products by HPLC as previously described (Laverty et al. 2019).

A non-limiting list of such cannabinoids is already quite established and some are provided infra. However, it is expected that during the life of a patent maturing from this application many relevant cannabinoids will be uncovered and the scope of the term cannabinoids is intended to include all such new cannabinoids a priori.

The classical cannabinoids are concentrated in a viscous resin produced in structures known as glandular trichomes. At least 143 different cannabinoids have been isolated from the Cannabis plant.

The best studied phytocannabinoids include tetrahydrocannabinol (THC), cannabidiol (CBD) and cannabinol (CBN).

Most classes derive from cannabigerol-type (CBG) compounds and differ mainly in the way this precursor is cyclized. The classical cannabinoids are derived from their respective 2-carboxylic acids (2-COOH) by decarboxylation (catalyzed by heat, light, or alkaline conditions).

THC (tetrahydrocannabinol)
THCA (tetrahydrocannabinolic acid)
CBD (cannabidiol)
CBDA (cannabidiolic acid)
CBN (cannabinol)
CBG (cannabigerol)
CBC (cannabichromene)
CBL (cannabicyclol)
CBV (cannabivarin)
THCV (tetrahydrocannabivarin)
CBDV (cannabidivarin)
CBCV (cannabichromevarin)
CBGV (cannabigerovarin)
CBGM (cannabigerol monomethyl ether)
CBE (cannabielsoin)
CBT (cannabicitran)

### Cannabinodiol (CBDL)

### Cannabigerol Monoethyl Ether (CBGM).

The term “plant” as used herein encompasses whole plants, a grafted plant, ancestors and progeny of the plants and plant parts, including flowers, trichomes, seeds, shoots, stems, roots, rootstock, scion, and plant cells, tissues and organs. The plant may be in any form including suspension cultures, embryos, meristematic regions, callus tissue, leaves, gametophytes, sporophytes, pollen, and microspores.

Plants that may be useful in the methods of the invention include all plants which belong to the superfamily Viridiplantee, in particular monocotyledonous and dicotyledonous plants.

The terms “cannabis” refers to the genus which includes all different species including Cannabis sativa, Cannabis indica and Cannabis ruderalis as well as wild Cannabis.

According to a specific embodiment, the Cannabis is Cannabis sativa.

Cannabis is diploid, having a chromosome complement of 2n=20, although polyploid individuals have been artificially produced and are also contemplated herein. The first genome sequence of Cannabis, which is estimated to be 820 Mb in size, was published in 2011.

All known strains of Cannabis are wind-pollinated and the fruit is an achene. Most strains of Cannabis are short day plants, with the possible exception of C. sativa subsp. sativa var. spontanea (=C. ruderalis), which is commonly described as “auto-flowering” and may be day-neutral.

Cannabis has long been used for drug and industrial purposes: fiber (hemp), for seed and seed oils, extracts for medicinal purposes, and as a recreational drug. The selected genetic background (e.g., cultivar) depends on the future use.

The term “variety” as used herein has identical meaning to the corresponding definition in the International Convention for the Protection of New Varieties of Plants (UPOV treaty), of Dec. 2, 1961, as Revised at Geneva on Nov. 10, 1972, on Oct. 23, 1978, and on Mar. 19, 1991. Thus, “variety” means a plant grouping within a single botanical taxon of the lowest known rank, which grouping, irrespective of whether the conditions for the grant of a breeder's right are fully met, can be i) defined by the expression of the characteristics resulting from a given genotype or combination of genotypes, ii) distinguished from any other plant grouping by the expression of at least one of the characteristics and iii) considered as a unit with regard to its suitability for being propagated unchanged.

The term “variety” is interchangeable with “cultivar”.

As mentioned, the method is effected by modulating expression in the cell, plant or part thereof of at least one polypeptide comprising an amino acid sequence at least 95% identical to SEQ ID NO: 1-15 and 18-86, the polypeptide being capable of modulating cannabinoid synthesis.

As used herein “modulating cannabinoid synthesis” means shifting or changing the natural occurring process in the cell, plant or part thereof in terms of cannabinoid profile as compared to the same genetic background without the modulation of the expression of the polypeptide as described herein (also referred to as “control”).

According to a specific embodiment, the modulating causes an increase in at least one cannabinoid in the modulated cell.

As used herein the term “increasing” or “increase” refers to at least about 2%, at least about 3%, at least about 4%, at least about 5%, at least about 10%, at least about 15%, at least about 20%, at least about 30%, at least about 40%, at least about 50%, at least about 60%, at least about 70%, at least about 80%, 2 fold, 5 fold, 10 fold, 100 fold increase in the cannabinoid as compared to a control plant (a plant which is not modified with the polynucleotide or polypeptides of the invention), such as a native plant, a wild type plant, a non-transformed plant or a non-genomic edited plant of the same species which is grown under the same (e.g., identical) growth conditions.

As used herein the term “decreasing” or “decrease” refers to at least about 2%, at least about 3%, at least about 4%, at least about 5%, at least about 10%, at least about 15%, at least about 20%, at least about 30%, at least about 40%, at least about 50%, at least about 60%, at least about 70%, at least about 80%, 2 fold, 5 fold, 10 fold, 100 fold decrease in the cannabinoid as compared to a control plant (a plant which is not modified with the polynucleotide or polypeptides of the invention), such as a native plant, a wild type plant, a non-transformed plant or a non-genomic edited plant of the same species which is grown under the same (e.g., identical) growth conditions.

The present inventors uncovered 8 groups of genes involved in cannabinoid synthesis.

According to an embodiment, these genes are cannabinoid synthases.

These are termed as follows:

Group 1: CBCAS-like genes (SEQ ID NOs: 87-101 for the polynucleotide sequences; and SEQ ID NOs: 1-15 for the polypeptide sequences).

Group 2: THCAS-like genes (SEQ ID NOs: 102-103 for the polynucleotide sequences; and SEQ ID NOs: 16-17 for the polypeptide sequences).

Group 3: (SEQ ID NOs: 104-133 for the polynucleotide sequences; and SEQ ID NOs: 18-47 for the polypeptide sequences).

Group 4: CBDAS-like genes (SEQ ID NOs: 134-141 for the polynucleotide sequences; and SEQ ID NOs: 48-55 for the polypeptide sequences).

Group 5: CBGAS-like genes (SEQ ID NOs: 142-149 for the polynucleotide sequences; and SEQ ID NOs: 56-63 for the polypeptide sequences).

Group 6: (SEQ ID NO: 150 for the polynucleotide sequence; and SEQ ID NOs: 64 for the polypeptide sequence).

Group 7: (SEQ ID NOs: 151-167 for the polynucleotide sequences; and SEQ ID NOs: 65-81 for the polypeptide sequences).

Group 8: (SEQ ID NOs: 168-172 for the polynucleotide sequences; and SEQ ID NOs: 82-86 for the polypeptide sequences).

Contemplated according to some embodiments are sequences with an upstream regulatory sequence. Also contemplated are the open reading frames without the regulatory sequences (starting from the ATG).

Also contemplated herein are homologs of these genes as further described hereinbelow.

The present teachings contemplate modulation of at least one (e.g., 2, 3, 4, 5) of the genes mentioned herein. These genes can be from the same group or from different group. According to some embodiments, when more than one gene is modulated then both can be upregulated, both can be downregulated, one can be upregulated while the other downregulated, each of which is considered a different embodiment.

Modulation of gene expression can be achieved by means of transgenesis, genome editing and especially in plants also by sexual breeding. Each of these options is considered a different embodiment.

According to one embodiment, modulation refers to upregulating expression of the polypeptide, also referred to as “over-expression”.

The F2,3 phrase “over-expressing a polypeptide” as used herein refers to increasing the level of the polypeptide within the plant as compared to a control plant of the same species under the same growth conditions.

According to some embodiments of the invention the increased level of the polypeptide is in a specific cell type or organ of the plant.

According to some embodiments of the invention, the increased level of the polypeptide is in a temporal time point of the plant.

Such a regulated gene expression when the shift in the cannabinoid profile is toxic to the plant or the cell.

According to some embodiments of the invention, the increased level of the polypeptide is during the whole life cycle of the plant.

For example, over-expression of a polypeptide can be achieved by elevating the expression level of a native gene of a plant as compared to a control plant. This can be done for example, by means of genome editing which are further described hereinunder, e.g., by introducing mutation(s) in regulatory element(s) (e.g., an enhancer, a promoter, an untranslated region, an intronic region) which result in upregulation of the native gene, and/or by Homology Directed Repair (HDR), e.g., for introducing a “repair template” encoding the polypeptide-of-interest.

Additionally and/or alternatively, over-expression of a polypeptide can be achieved by increasing a level of a polypeptide-of-interest due to expression of a heterologous polynucleotide by means of recombinant DNA technology, e.g., using a nucleic acid construct comprising a polynucleotide encoding the polypeptide-of-interest.

It should be noted that in case the plant-of-interest (e.g., a plant for which over-expression of a polypeptide is desired) has no detectable expression level of the polypeptide-of-interest prior to employing the method of some embodiments of the invention, qualifying an “over-expression” of the polypeptide in the plant is performed by determination of a positive detectable expression level of the polypeptide-of-interest in a plant cell and/or a plant.

Additionally and/or alternatively in case the plant-of-interest (e.g., a plant for which over-expression of a polypeptide is desired) has some degree of detectable expression level of the polypeptide-of-interest prior to employing the method of some embodiments of the invention, qualifying an “over-expression” of the polypeptide in the plant is performed by determination of an increased level of expression of the polypeptide-of-interest in a plant cell and/or a plant as compared to a control plant cell and/or plant, respectively, of the same species which is grown under the same (e.g., identical) growth conditions.

Methods of detecting presence or absence of a polypeptide in a plant cell and/or in a plant, as well as quantification of protein expression levels are well known in the art (e.g., protein detection methods), and are further described hereinunder.

As used herein the phrase “expressing an exogenous polynucleotide encoding a polypeptide” refers to expression at the mRNA level.

As used herein the phrase “expressing an exogenous polynucleotide encoding a polypeptide” refers to expression at the mRNA level.

As used herein, the phrase “exogenous polynucleotide” refers to a heterologous nucleic acid sequence which may not be naturally expressed within the plant (e.g., a nucleic acid sequence from a different species) or which overexpression in the plant is desired. The exogenous polynucleotide may be introduced into the plant in a stable or transient manner, so as to produce a ribonucleic acid (RNA) molecule and/or a polypeptide molecule. It should be noted that the exogenous polynucleotide may comprise a nucleic acid sequence which is identical or partially homologous to an endogenous nucleic acid sequence of the plant.

The term “endogenous” as used herein refers to any polynucleotide or polypeptide which is present and/or naturally expressed within a plant or a cell thereof.

According to some embodiments of the invention, the exogenous polynucleotide of the invention comprises a nucleic acid sequence encoding a polypeptide having an amino acid sequence about 90%, at least about 91%, at least about 92%, at least about 93%, at least about 94%, at least about 95%, at least about 96%, at least about 97%, at least about 98%, at least about 99%, or more say 100% identity to the amino acid sequence selected from the group consisting of SEQ ID NOs: 1-15 and 18-86.

According to some embodiments of the invention, the exogenous polynucleotide of the invention comprises a nucleic acid sequence encoding a polypeptide having an amino acid sequence at least about 95%, at least about 96%, at least about 97%, at least about 98%, at least about 99%, or more say 100% identity to the amino acid sequence selected from the group consisting of SEQ ID NOs: 1-15 and 18-86.

According to some embodiments of the invention, the exogenous polynucleotide of the invention comprises a nucleic acid sequence encoding a polypeptide having an amino acid sequence at least about 99%, at least about 99.5%, or more say 100% identity to the amino acid sequence selected from the group consisting of SEQ ID NOs: 1-15 and 18-86.

Homologous sequences include both orthologous and paralogous sequences. The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species (Koonin E V and Galperin M Y (Sequence—Evolution—Function: Computational Approaches in Comparative Genomics. Boston: Kluwer Academic; 2003. Chapter 2, Evolutionary Concept in Genetics and Genomics. Available from: ncbi (dot) nlm (dot) nih (dot) gov/books/NBK20255) and therefore have great likelihood of having the same function.

One option to identify orthologues in monocot plant species is by performing a reciprocal blast search. This may be done by a first blast involving blasting the sequence-of-interest against any sequence database, such as the publicly available NCBI database which may be found at: ncbi (dot) nlm (dot) nih (dot) gov. If orthologues in rice were sought, the sequence-of-interest would be blasted against, for example, the 28,469 full-length cDNA clones from Oryza sativa Nipponbare available at NCBI. The blast results may be filtered. The full-length sequences of either the filtered results or the non-filtered results are then blasted back (second blast) against the sequences of the organism from which the sequence-of-interest is derived. The results of the first and second blasts are then compared. An orthologue is identified when the sequence resulting in the highest score (best hit) in the first blast identifies in the second blast the query sequence (the original sequence-of-interest) as the best hit. Using the same rational a paralogue (homolog to a gene in the same organism) is found. In case of large sequence families, the ClustalW program may be used [ebi (dot) ac (dot) uk/Tools/clustalw2/index (dot) html], followed by a neighbor-joining tree (wikipedia (dot) org/wiki/Neighbor-joining) which helps visualizing the clustering.

Homology (e.g., percent homology, sequence identity+sequence similarity) can be determined using any homology comparison software computing a pairwise sequence alignment.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

Identity (e.g., percent homology) can be determined using any homology comparison software, including for example, the BlastN software of the National Center of Biotechnology Information (NCBI) such as by using default parameters.

According to some embodiments of the invention, the identity is a global identity, i.e., an identity over the entire amino acid or nucleic acid sequences of the invention and not over portions thereof.

According to some embodiments of the invention, the term “homology” or “homologous” refers to identity of two or more nucleic acid sequences; or identity of two or more amino acid sequences; or the identity of an amino acid sequence to one or more nucleic acid sequence.

According to some embodiments of the invention, the homology is a global homology, i.e., an homology over the entire amino acid or nucleic acid sequences of the invention and not over portions thereof.

The degree of homology or identity between two or more sequences can be determined using various known sequence comparison tools. Following is a non-limiting description of such tools which can be used along with some embodiments of the invention.

Pairwise global alignment was defined by S. B. Needleman and C. D. Wunsch, “A general method applicable to the search of similarities in the amino acid sequence of two proteins” Journal of Molecular Biology, 1970, pages 443-53, volume 48).

For example, when starting from a polypeptide sequence and comparing to other polypeptide sequences, the EMBOSS-6.0.1 Needleman-Wunsch algorithm (available from emboss(dot)sourceforge(dot)net/apps/cvs/emboss/apps/needle(dot)html) can be used to find the optimum alignment (including gaps) of two sequences along their entire length—a “Global alignment”. Default parameters for Needleman-Wunsch algorithm (EMBOSS-6.0.1) include: gapopen=10; gapextend=0.5; datafile=EBLOSUM62; brief=YES.

According to some embodiments of the invention, the parameters used with the EMBOSS-6.0.1 tool (for protein-protein comparison) include: gapopen=8; gapextend=2; datafile=EBLOSUM62; brief=YES.

According to some embodiments of the invention, the threshold used to determine homology using the EMBOSS-6.0.1 Needleman-Wunsch algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

When starting from a polypeptide sequence and comparing to polynucleotide sequences, the OneModel FramePlus algorithm [Halperin, E., Faigler, S. and Gill-More, R. (1999)—FramePlus: aligning DNA to protein sequences. Bioinformatics, 15, 867-873) (available from biocceleration(dot)com/Products(dot)html] can be used with following default parameters: model=frame+_p2n.model mode=local.

According to some embodiments of the invention, the parameters used with the OneModel FramePlus algorithm are model=frame+_p2n.model, mode=qglobal.

According to some embodiments of the invention, the threshold used to determine homology using the OneModel FramePlus algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

When starting with a polynucleotide sequence and comparing to other polynucleotide sequences the EMBOSS-6.0.1 Needleman-Wunsch algorithm (available from emboss(dot)sourceforge(dot)net/apps/cvs/emboss/apps/needle(dot)html) can be used with the following default parameters: (EMBOSS-6.0.1) gapopen=10; gapextend=0.5; datafile=EDNAFULL; brief=YES.

According to some embodiments of the invention, the parameters used with the EMBOSS-6.0.1 Needleman-Wunsch algorithm are gapopen=10; gapextend=0.2; datafile=EDNAFULL; brief=YES.

According to some embodiments of the invention, the threshold used to determine homology using the EMBOSS-6.0.1 Needleman-Wunsch algorithm for comparison of polynucleotides with polynucleotides is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiment, determination of the degree of homology further requires employing the Smith-Waterman algorithm (for protein-protein comparison or nucleotide-nucleotide comparison).

Default parameters for GenCore 6.0 Smith-Waterman algorithm include: model=sw.model.

According to some embodiments of the invention, the threshold used to determine homology using the Smith-Waterman algorithm is 80%, 81%, 82%, 83%, 84%, 85%, 86%, 87%, 88%, 89%, 90%, 91%, 92%, 93%, 94%, 95%, 96%, 97%, 98%, 99%, or 100%.

According to some embodiments of the invention, the global homology is performed on sequences which are pre-selected by local homology to the polypeptide or polynucleotide of interest (e.g., 95% identity over 60% of the sequence length), prior to performing the global homology to the polypeptide or polynucleotide of interest (e.g., 95% global homology on the entire sequence). For example, homologous sequences are selected using the BLAST software with the Blastp and tBlastn algorithms as filters for the first stage, and the needle (EMBOSS package) or Frame+ algorithm alignment for the second stage. Local identity (Blast alignments) is defined with a very permissive cutoff—95% Identity on a span of 60% of the sequences lengths because it is used only as a filter for the global alignment stage. In this specific embodiment (when the local identity is used), the default filtering of the Blast package is not utilized (by setting the parameter “-F F”).

In the second stage, homologs are defined based on a global identity of at least 95% or 99% to the core gene polypeptide sequence.

According to some embodiments of the invention, the exogenous polynucleotide of the invention encodes a polypeptide as described herein.

According to some embodiments of the invention, the exogenous polynucleotide encodes a polypeptide consisting of the amino acid sequence set forth by SEQ ID NO: 1-15 and 18-86.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

The term “isolated” refers to at least partially separated from the natural environment e.g., from a plant cell.

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

Nucleic acid sequences encoding the polypeptides of the present invention may be optimized for expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn−Yn)/Yn] 2/N, where Xn refers to the frequency of usage of codon n in highly expressed plant genes, where Yn to the frequency of usage of codon n in the gene of interest and N refers to the total number of codons in the gene of interest. A Table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization Tables such as those provided on-line at the Codon Usage Database through the NIAS (National Institute of Agrobiological Sciences) DNA bank in Japan (kazusa (dot) or (dot) jp/codon/). The Codon Usage Database contains codon usage tables for a number of different species, with each codon usage Table having been statistically determined based on the data present in Genbank.

By using the above Tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Thus, the invention encompasses nucleic acid sequences described hereinabove; fragments thereof, sequences hybridizable therewith, sequences homologous thereto, sequences encoding similar polypeptides with different codon usage, altered sequences characterized by mutations, such as deletion, insertion or substitution of one or more nucleotides, either naturally occurring or man induced, either randomly or in a targeted fashion.

According to some embodiments of the invention, the exogenous polynucleotide encodes a polypeptide comprising an amino acid sequence at least about 90%, at least about 91%, at least about 92%, at least about 93%, at least about 93%, at least about 94%, at least about 95%, at least about 96%, at least about 97%, at least about 98%, at least about 99%, e.g., 100% identical to the amino acid sequence of a naturally occurring plant orthologue of the polypeptide selected from the group consisting of SEQ ID NOs: 1-15 and 18-86.

According to some embodiments of the invention, the polypeptide comprising an amino acid sequence at least 95%, at least about 96%, at least about 97%, at least about 98%, at least about 99%, e.g., 100% identical to the amino acid sequence of a naturally occurring plant orthologue of the polypeptide selected from the group consisting of SEQ ID NOs: 1-15 and 18-86.

According to some embodiments of the invention, the polypeptide comprising an amino acid sequence at least about 99%, e.g., 100% identical to the amino acid sequence of a naturally occurring plant orthologue of the polypeptide selected from the group consisting of SEQ ID NOs: 1-15 and 18-86.

The invention provides an isolated polynucleotide comprising a nucleic acid sequence at least about 90%, at least about 91%, at least about 92%, at least about 93%, at least about 93%, at least about 94%, at least about 95%, at least about 96%, at least about 97%, at least about 98%, at least about 99%, e.g., 100% identical to the polynucleotide selected from the group consisting of SEQ ID NOs: 87-101 and 104-173.

The invention provides an isolated polynucleotide comprising a nucleic acid sequence at least about 95%, at least about 96%, at least about 97%, at least about 98%, at least about 99%, e.g., 100% identical to the polynucleotide selected from the group consisting of SEQ ID NOs: 87-101 and 104-173.

The invention provides an isolated polynucleotide comprising a nucleic acid sequence at least about 99%, 99.5% e.g., 100% identical to the polynucleotide selected from the group consisting of SEQ ID NOs: 87-101 and 104-173.

According to some embodiments of the invention the nucleic acid sequence (or actually polypeptide encoded thereby) is capable of modulating cannabis synthesis. In other words affecting the cannabinoid profile of the plant or cell.

Downregulation (gene silencing) of the transcription or translation product of an endogenous gene can be achieved by co-suppression, antisense suppression, RNA intereference and ribozyme molecules.

Co-suppression (sense suppression)—Inhibition of the endogenous gene can be achieved by co-suppression, using an RNA molecule (or an expression vector encoding same) which is in the sense orientation with respect to the transcription direction of the endogenous gene. The polynucleotide used for co-suppression may correspond to all or part of the sequence encoding the endogenous polypeptide and/or to all or part of the 5′ and/or 3′ untranslated region of the endogenous transcript; it may also be an unpolyadenylated RNA; an RNA which lacks a 5′ cap structure; or an RNA which contains an unsplicable intron. In some embodiments, the polynucleotide used for co-suppression is designed to eliminate the start codon of the endogenous polynucleotide so that no protein product will be translated. Methods of co-suppression using a full-length cDNA sequence as well as a partial cDNA sequence are known in the art (see, for example, U.S. Pat. No. 5,231,020).

According to some embodiments of the invention, downregulation of the endogenous gene is performed using an amplicon expression vector which comprises a plant virus-derived sequence that contains all or part of the target gene but generally not all of the genes of the native virus. The viral sequences present in the transcription product of the expression vector allow the transcription product to direct its own replication. The transcripts produced by the amplicon may be either sense or antisense relative to the target sequence [see for example, Angell and Baulcombe, (1997) EMBO J. 16:3675-3684; Angell and Baulcombe, (1999) Plant J. 20:357-362, and U.S. Pat. No. 6,646,805, each of which is herein incorporated by reference].

Antisense suppression—Antisense suppression can be performed using an antisense polynucleotide or an expression vector which is designed to express an RNA molecule complementary to all or part of the messenger RNA (mRNA) encoding the endogenous polypeptide and/or to all or part of the 5′ and/or 3′ untranslated region of the endogenous gene. Over expression of the antisense RNA molecule can result in reduced expression of the native (endogenous) gene. The antisense polynucleotide may be fully complementary to the target sequence (i.e., 100% identical to the complement of the target sequence) or partially complementary to the target sequence (i.e., less than 100% identical, e.g., less than 90%, less than 80% identical to the complement of the target sequence). Antisense suppression may be used to inhibit the expression of multiple proteins in the same plant (see e.g., U.S. Pat. No. 5,942,657). In addition, portions of the antisense nucleotides may be used to disrupt the expression of the target gene. Generally, sequences of at least about 50 nucleotides, at least about 100 nucleotides, at least about 200 nucleotides, at least about 300, at least about 400, at least about 450, at least about 500, at least about 550, or greater may be used. Methods of using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal [See, U.S. Patent Publication No. 20020048814, herein incorporated by reference].

RNA intereference—RNA intereference can be achieved using a polynucleotide, which can anneal to itself and form a double stranded RNA having a stem-loop structure (also called hairpin structure), or using two polynucleotides, which form a double stranded RNA.

For hairpin RNA (hpRNA) interference, the expression vector is designed to express an RNA molecule that hybridizes to itself to form a hairpin structure that comprises a single-stranded loop region and a base-paired stem.

In some embodiments of the invention, the base-paired stem region of the hpRNA molecule determines the specificity of the RNA interference. In this configuration, the sense sequence of the base-paired stem region may correspond to all or part of the endogenous mRNA to be downregulated, or to a portion of a promoter sequence controlling expression of the endogenous gene to be inhibited; and the antisense sequence of the base-paired stem region is fully or partially complementary to the sense sequence. Such hpRNA molecules are highly efficient at inhibiting the expression of endogenous genes, in a manner which is inherited by subsequent generations of plants [See, e.g., Chuang and Meyerowitz, (2000) Proc. Natl. Acad. Sci. USA 97:4985-4990; Stoutjesdijk, et al., (2002) Plant Physiol. 129:1723-1731; and Waterhouse and Helliwell, (2003) Nat. Rev. Genet. 4:29-38; Chuang and Meyerowitz, (2000) Proc. Natl. Acad. Sci. USA 97:4985-4990; Pandolfini et al., BMC Biotechnology 3:7; Panstruga, et al., (2003) Mol. Biol. Rep. 30:135-140; and U.S. Patent Publication No. 2003/0175965; each of which is incorporated by reference].

According to some embodiments of the invention, the sense sequence of the base-paired stem is from about 10 nucleotides to about 2,500 nucleotides in length, e.g., from about 10 nucleotides to about 500 nucleotides, e.g., from about 15 nucleotides to about 300 nucleotides, e.g., from about 20 nucleotides to about 100 nucleotides, e.g., or from about 25 nucleotides to about 100 nucleotides.

According to some embodiments of the invention, the antisense sequence of the base-paired stem may have a length that is shorter, the same as, or longer than the length of the corresponding sense sequence.

According to some embodiments of the invention, the loop portion of the hpRNA can be from about 10 nucleotides to about 500 nucleotides in length, for example from about 15 nucleotides to about 100 nucleotides, from about 20 nucleotides to about 300 nucleotides or from about 25 nucleotides to about 400 nucleotides in length.

According to some embodiments of the invention, the loop portion of the hpRNA can include an intron (ihpRNA), which is capable of being spliced in the host cell. The use of an intron minimizes the size of the loop in the hairpin RNA molecule following splicing and thus increases efficiency of the interference [See, for example, Smith, et al., (2000) Nature 407:319-320; Wesley, et al., (2001) Plant J. 27:581-590; Wang and Waterhouse, (2001) Curr. Opin. Plant Biol. 5:146-150; Helliwell and Waterhouse, (2003) Methods 30:289-295; Brummell, et al. (2003) Plant J. 33:793-800; and U.S. Patent Publication No. 2003/0180945; WO 98/53083; WO 99/32619; WO 98/36083; WO 99/53050; US 20040214330; US 20030180945; U.S. Pat. Nos. 5,034,323; 6,452,067; 6,777,588; 6,573,099 and 6,326,527; each of which is herein incorporated by reference].

In some embodiments of the invention, the loop region of the hairpin RNA determines the specificity of the RNA interference to its target endogenous RNA. In this configuration, the loop sequence corresponds to all or part of the endogenous messenger RNA of the target gene. See, for example, WO 02/00904; Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., Curr. Biol. (2001) 11:436-440), each of which is incorporated herein by reference.

For double-stranded RNA (dsRNA) interference, the sense and antisense RNA molecules can be expressed in the same cell from a single expression vector (which comprises sequences of both strands) or from two expression vectors (each comprising the sequence of one of the strands). Methods for using dsRNA interference to inhibit the expression of endogenous plant genes are described in Waterhouse, et al., (1998) Proc. Natl. Acad. Sci. USA 95:13959-13964; and WO 99/49029, WO 99/53050, WO 99/61631, and WO 00/49035; each of which is herein incorporated by reference.

According to some embodiments of the invention, RNA interference is effected using an expression vector designed to express an RNA molecule that is modeled on an endogenous micro RNAs (miRNA) gene. Micro RNAs (miRNAs) are regulatory agents consisting of about 22 ribonucleotides and highly efficient at inhibiting the expression of endogenous genes [Javier, et al., (2003) Nature 425:257-263]. The miRNA gene encodes an RNA that forms a hairpin structure containing a 22-nucleotide sequence that is complementary to the endogenous target gene.

Ribozyme—Catalytic RNA molecules, ribozymes, are designed to cleave particular mRNA transcripts, thus preventing expression of their encoded polypeptides. Ribozymes cleave mRNA at site-specific recognition sequences. For example, “hammerhead ribozymes” (see, for example, U.S. Pat. No. 5,254,678) cleave mRNAs at locations dictated by flanking regions that form complementary base pairs with the target mRNA. The sole requirement is that the target RNA contains a 5′-UG-3′ nucleotide sequence. Hammerhead ribozyme sequences can be embedded in a stable RNA such as a transfer RNA (tRNA) to increase cleavage efficiency in vivo [Perriman et al. (1995) Proc. Natl. Acad. Sci. USA, 92(13):6175-6179; de Feyter and Gaudron Methods in Molecular Biology, Vol. 74, Chapter 43, “Expressing Ribozymes in Plants”, Edited by Turner, P. C, Humana Press Inc., Totowa, N.J.; U.S. Pat. No. 6,423,885]. RNA endoribonucleases such as that found in Tetrahymena thermophila are also useful ribozymes (U.S. Pat. No. 4,987,071).

Genome editing can also be used as mentioned hereinabove for over-expression (gain of function) or downregulation (loss of function).

Genome editing is a powerful mean to impact target traits by modifications of the target plant genome sequence. Such modifications can result in new or modified alleles or regulatory elements. Thus, genome editing employs reverse genetics by artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Since most genome-editing techniques can leave behind minimal traces of DNA alterations evident in a small number of nucleotides as compared to transgenic plants, crops created through gene editing could avoid the stringent regulation procedures commonly associated with genetically modified (GM) crop development. On the other hand, the traces of genome-edited techniques can be used for marker assisted selection (MAS) as is further described hereinunder. Target plants for the mutagenesis/genome editing methods according to the invention are any plants of interest including monocot or dicot plants.

Over expression of a polypeptide by genome editing can be achieved by: (i) replacing an endogenous sequence encoding the polypeptide of interest or a regulatory sequence under the control which it is placed, and/or (ii) inserting a new gene encoding the polypeptide of interest in a targeted region of the genome, and/or (iii) introducing point mutations which result in upregulation of the gene encoding the polypeptide of interest (e.g., by altering the regulatory sequences such as promoter, enhancers, 5′-UTR and/or 3′-UTR, or mutations in the coding sequence).

Homology Directed Repair (HDR)

Homology Directed Repair (HDR) can be used to generate specific nucleotide changes (also known as gene “edits”) ranging from a single nucleotide change to large insertions. In order to utilize HDR for gene editing, a DNA “repair template” containing the desired sequence must be delivered into the cell type of interest with the guide RNA [gRNA(s)] and Cas9 or Cas9 nickase. The repair template must contain the desired edit as well as additional homologous sequence immediately upstream and downstream of the target (termed left and right homology arms). The length and binding position of each homology arm is dependent on the size of the change being introduced. The repair template can be a single stranded oligonucleotide, double-stranded oligonucleotide, or double-stranded DNA plasmid depending on the specific application. It is worth noting that the repair template must lack the Protospacer Adjacent Motif (PAM) sequence that is present in the genomic DNA, otherwise the repair template becomes a suitable target for Cas9 cleavage. For example, the PAM could be mutated such that it is no longer present, but the coding region of the gene is not affected (i.e. a silent mutation).

The efficiency of HDR is generally low (<10% of modified alleles) even in cells that express Cas9, gRNA and an exogenous repair template. For this reason, many laboratories are attempting to artificially enhance HDR by synchronizing the cells within the cell cycle stage when HDR is most active, or by chemically or genetically inhibiting genes involved in Non-Homologous End Joining (NHEJ). The low efficiency of HDR has several important practical implications. First, since the efficiency of Cas9 cleavage is relatively high and the efficiency of HDR is relatively low, a portion of the Cas9-induced double strand breaks (DSBs) will be repaired via NHEJ. In other words, the resulting population of cells will contain some combination of wild-type alleles, NHEJ-repaired alleles, and/or the desired HDR-edited allele. Therefore, it is important to confirm the presence of the desired edit experimentally, and if necessary, isolate clones containing the desired edit.

The HDR method was successfully used for targeting a specific modification in a coding sequence of a gene in plants (Budhagatapalli Nagaveni et al. 2015. “Targeted Modification of Gene Function Exploiting Homology-Directed Repair of TALEN-Mediated Double-Strand Breaks in Barley”. G3 (Bethesda). 2015 September; 5(9): 1857-1863). Thus, the gfp-specific transcription activator-like effector nucleases were used along with a repair template that, via HDR, facilitates conversion of gfp into yfp, which is associated with a single amino acid exchange in the gene product. The resulting yellow-fluorescent protein accumulation along with sequencing confirmed the success of the genomic editing.

Similarly, Zhao Yongping et al. 2016 (An alternative strategy for targeted gene replacement in plants using a dual-sgRNA/Cas9 design. Scientific Reports 6, Article number: 23890 (2016)) describe co-transformation of Arabidopsis plants with a combinatory dual-sgRNA/Cas9 vector that successfully deleted miRNA gene regions (MIR169a and MIR827a) and second construct that contains sites homologous to Arabidopsis TERMINAL FLOWER 1 (TFL1) for homology-directed repair (HDR) with regions corresponding to the two sgRNAs on the modified construct to provide both targeted deletion and donor repair for targeted gene replacement by HDR.

Activation of Target Genes Using CRISPR/Cas9

Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of CRISPR-associated endonuclease (Cas9) in conjunction with synthetic gRNAs can be used to produce targeted double-stranded brakes in a variety of different species.

The CRISPR/Cas9 system is a remarkably flexible tool for genome manipulation. A unique feature of Cas9 is its ability to bind target DNA independently of its ability to cleave target DNA. Specifically, both RuvC- and HNH-nuclease domains can be rendered inactive by point mutations (D10A and H840A in SpCas9), resulting in a nuclease dead Cas9 (dCas9) molecule that cannot cleave target DNA. The dCas9 molecule retains the ability to bind to target DNA based on the gRNA targeting sequence. The dCas9 can be tagged with transcriptional activators, and targeting these dCas9 fusion proteins to the promoter region results in robust transcription activation of downstream target genes. The simplest dCas9-based activators consist of dCas9 fused directly to a single transcriptional activator. Importantly, unlike the genome modifications induced by Cas9 or Cas9 nickase, dCas9-mediated gene activation is reversible, since it does not permanently modify the genomic DNA.

Indeed, genome editing was successfully used to over-express a protein of interest in a plant by, for example, mutating a regulatory sequence, such as a promoter to overexpress the endogenous polynucleotide operably linked to the regulatory sequence. For example, U.S. Patent Application Publication No. 20160102316 to Rubio Munoz, Vicente et al. which is fully incorporated herein by reference, describes plants with increased expression of an endogenous DDA1 plant nucleic acid sequence wherein the endogenous DDA1 promoter carries a mutation introduced by mutagenesis or genome editing which results in increased expression of the DDA1 gene, using for example, CRISPR. The method involves targeting of Cas9 to the specific genomic locus, in this case DDA1, via a 20 nucleotide guide sequence of the single-guide RNA. An online CRISPR Design Tool can identify suitable target sites (www(dot)tools(dot)genome-engineering(dot)org. Ran et al. Genome engineering using the CRISPR-Cas9 system nature protocols, VOL. 8 NO. 11, 2281-2308, 2013).

The CRISPR-Cas system was used for altering gene expression in plants as described in U.S. Patent Application publication No. 20150067922 to Yang; Yinong et al., which is fully incorporated herein by reference. Thus, the engineered, non-naturally occurring gene editing system comprises two regulatory elements, wherein the first regulatory element (a) operable in a plant cell operably linked to at least one nucleotide sequence encoding a CRISPR-Cas system guide RNA (gRNA) that hybridizes with the target sequence in the plant, and a second regulatory element (b) operable in a plant cell operably linked to a nucleotide sequence encoding a Type-II CRISPR-associated nuclease, wherein components (a) and (b) are located on same or different vectors of the system, whereby the guide RNA targets the target sequence and the CRISPR-associated nuclease cleaves the DNA molecule, thus altering the expression of a gene product in a plant. It should be noted that the CRISPR-associated nuclease and the guide RNA do not naturally occur together.

In addition, as described above, point mutations which activate a gene-of-interest and/or which result in over-expression of a polypeptide-of-interest can be also introduced into plants by means of genome editing. Such mutation can be for example, deletions of repressor sequences which result in activation of the gene-of-interest; and/or mutations which insert nucleotides and result in activation of regulatory sequences such as promoters and/or enhancers.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8,129,134; 8,133,697; 8,143,015; 8,143,016; 8,148,098; or 8,163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the FokI domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

The CRIPSR/Cas system for genome editing contains two distinct components: a gRNA and an endonuclease e.g. Cas9.

The gRNA is typically a 20 nucleotide sequence encoding a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. Dec. 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

According to some embodiments of the invention, there is provided a plant cell exogenously expressing the polynucleotide of some embodiments of the invention, the nucleic acid construct of some embodiments of the invention and/or the polypeptide of some embodiments of the invention.

According to some embodiments of the invention, modulating expression t is effected by transforming one or more cells of the plant with the polynucleotide, followed by generating a mature plant from the transformed cells and cultivating the mature plant under conditions suitable for modulating the exogenous polynucleotide within the mature plant.

According to some embodiments of the invention, the transformation is effected by introducing to the plant cell a nucleic acid construct which includes the exogenous polynucleotide of some embodiments of the invention and at least one promoter for directing transcription of the exogenous polynucleotide in a host cell (a plant cell). Further details of suitable transformation approaches are provided hereinbelow.

As mentioned, the nucleic acid construct according to some embodiments of the invention comprises a promoter sequence and the isolated polynucleotide of some embodiments of the invention.

According to some embodiments of the invention, the isolated polynucleotide is operably linked to the promoter sequence.

A coding nucleic acid sequence is “operably linked” to a regulatory sequence (e.g., promoter) if the regulatory sequence is capable of exerting a regulatory effect on the coding sequence linked thereto.

As used herein, the term “promoter” refers to a region of DNA which lies upstream of the transcriptional initiation site of a gene to which RNA polymerase binds to initiate transcription of RNA. The promoter controls where (e.g., which portion of a plant) and/or when (e.g., at which stage or condition in the lifetime of an organism) the gene is expressed.

According to some embodiments of the invention, the promoter is heterologous to the isolated polynucleotide and/or to the host cell.

As used herein the phrase “heterologous promoter” refers to a promoter from a different species with respect to the species from which the polynucleotide is isolated, or to a promoter from the same species but from a different gene locus within the plant's genome with respect to the gene locus from which the polynucleotide sequence is isolated.

According to some embodiments of the invention, the isolated polynucleotide is heterologous to the plant cell (e.g., the polynucleotide is derived from a different plant species when compared to the plant cell, thus the isolated polynucleotide and the plant cell are not from the same plant species).

Any suitable promoter sequence can be used by the nucleic acid construct of the present invention. Preferably the promoter is a constitutive promoter, a tissue-specific, or a stress-inducible promoter.

According to some embodiments of the invention, the promoter is a plant promoter, which is suitable for expression of the exogenous polynucleotide in a plant cell.

The nucleic acid construct of some embodiments of the invention can be utilized to transform plant cells.

Constructs useful in the methods according to some embodiments of the invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein said nucleic acid sequence is operably linked to one or more regulatory sequences allowing expression in the plant cells.

In a particular embodiment of some embodiments of the invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of preferred promoters useful for the methods of some embodiments of the invention are presented in Table I, II, III.

TABLE I
Exemplary constitutive promoters for use in the
performance of some embodiments of the invention

| | Expression | |
|---|---|---|
| Gene Source | Pattern | Reference |
| Actin | constitutive | McElroy et al., Plant Cell, |
| | | 2:163-171, 1990 |
| CAMV 35S | constitutive | Odell et al., Nature, |
| | | 313:810-812, 1985 |
| CaMV 19S | constitutive | Nilsson et al., Physiol. |
| | | Plant 100:456-462, 1997 |
| GOS2 | constitutive | de Pater et al., Plant J |
| | | Nov;2(6):837-44, 1992 |
| ubiquitin | constitutive | Christensen et al., Plant |
| | | Mol. Biol. 18:675-689, 1992 |
| Rice cyclophilin | constitutive | Bucholz et al., Plant Mol |
| | | Biol. 25(5):837-43, 1994 |
| Maize H3 histone | constitutive | Lepetit et al., Mol. Gen. |
| | | Genet. 231:276-285, 1992 |
| Actin 2 | constitutive | An et al., Plant J. |
| | | 10(1);107-121, 1996 |

TABLE II
Exemplary seed-preferred promoters for use in the performance of some embodiments of the invention

| | Expression | |
|---|---|---|
| Gene Source | Pattern | Reference |
| Seed specific genes | seed | Simon, et al., Plant Mol. Biol. 5. 191, 1985; |
| | | Scofield, et al., J. Biol. Chem. 262:12202, 1987.; |
| | | Baszczynski, et al., Plant Mol. Biol. 14:633, 1990. |
| Brazil Nut albumin | seed | Pearson' et al., Plant Mol. Biol. 18:235-245, 1992. |
| legumin | seed | Ellis, et al. Plant Mol. Biol. 10:203-214, 1988 |
| Glutelin (rice) | seed | Takaiwa, et al., Mol. Gen. Genet. 208:15-22, 1986; |
| | | Takaiwa, et al., FEBS Letts. 221:43-47, 1987 |
| Zein | seed | Matzke et al. Plant Mol Biol, 143).323-32 1990 |
| napA | seed | Stalberg, et al., Planta 199:515-519, 1996 |
| wheat LMW and HMW, glutenin-1 | endosperm | Mol Gen Genet 216:81-15 and 18-86, 1989; |
| | | NAR 17:461-2, |
| Wheat SPA | seed | Albanietal, Plant Cell, 9:171-184, 1997 |
| wheat a, b and g gliadins | endosperm | EMBO3:1409-15, 1984 |
| Barley ltrl promoter | endosperm | |
| barley B1, C, D hordein | endosperm | Theor Appl Gen 98:1253-62, 1999; Plant J 4:343-55, |
| | | 1993; Mol Gen Genet 250:750-60, 1996 |
| Barley DOF | endosperm | Mena et al., The Plant Journal, 116(1):53-62, 1998 |
| Biz2 | endosperm | EP99106056.7 |
| Synthetic promoter | endosperm | Vicente-Carbajosa et al., Plant J. 13:629-640, 1998 |
| rice prolamin NRP33 | endosperm | Wu et al., Plant Cell Physiology 39(8) 885-889, 1998 |
| rice -globulin Glb-1 | endosperm | Wu et al., Plant Cell Physiology 398) 885-889, 1998 |
| rice OSH1 | emryo | Sato et al., Proc. Nati. Acad. Sci. USA, 93:8117-8122 |
| rice alpha-globulin REB/OHP-1 | endosperm | Nakase et al. Plant Mol. Biol. 33:513-S22, 1997 |
| rice ADP-glucose PP | endosperm | Trans Res 6:157-68, 1997 |
| maize ESR gene family | endosperm | Plant J 12:235-46, 1997 |
| sorgum gamma- kafirin | endosperm | PMB 32:1029-35, 1996 |
| KNOX | emryo | Postma-Haarsma et al., Plant Mol. Biol. 39:257-71, 1999 |
| rice oleosin | Embryo and aleuton | Wu et at, J. Biochem., 123:386, 1998 |
| sunflower oleosin | Seed (embryo and dry seed) | Cummins, et al., Plant Mol. Biol. 19:873-876, 1992 |

TABLE III
Exemplary flower-specific promoters for
use in the performance of the invention

| | Expression | |
|---|---|---|
| Gene Source | Pattern | Reference |
| AtPRP4 | flowers | www(dot)salus(dot) |
| | | Medium(dot)edu/mmg/tierney/html |
| chalene synthase | flowers | Van der Meer, et al., Plant |
| (chsA) | | Mol. Biol. 15, 95-109, 1990. |
| LAT52 | anther | Twell et al. Mol. Gen |
| | | Genet. 217:240-245 (1989) |
| apetala- 3 | flowers | |

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Once cells, plants or parts of having been modified to upregulate or downregulate the gene of interest, these are selected to find the genomic event and/or the requested phenotype.

Thus, according to an aspect of the invention there is provided a method of selecting a plant for a cannabinoid profile, the method comprising analyzing in the plant or part thereof presence of a nucleic acid sequence at least 95% identical to SEQ ID NO: 87-101 and 104-173 or amino acid sequence at least 95% identical to SEQ ID NO: 1-15 and 18-86, wherein presence or absence of the nucleic acid sequence or amino acid sequence is indicative of the cannabinoid profile.

Marker-assisted selection (MAS) can be used to identify the modification e.g., presence of an Indel following genome editing.

The sequence information and annotations uncovered by the present teachings can be harnessed in favor of uncovering the requested genotype and/or classical breeding. Thus, sub-sequence data of those polynucleotides described above, can be used as markers for marker assisted selection (MAS), in which a marker is used for indirect selection of a genetic determinant or determinants of a cannabinoid profile. Nucleic acid data of the present teachings (DNA or RNA sequence) may contain or be linked to polymorphic sites or genetic markers on the genome such as restriction fragment length polymorphism (RFLP), microsatellites and single nucleotide polymorphism (SNP), DNA fingerprinting (DFP), amplified fragment length polymorphism (AFLP), expression level polymorphism, polymorphism of the encoded polypeptide and any other polymorphism at the DNA or RNA sequence.

Alternatively or additionally the method comprises determining the cannabinoid profile or a specific cannabinoid of the plant or part thereof or cell.

Diverse chromatographic techniques have been used purify cannabinoid compounds from the plant Cannabis sativa. For example, Flash chromatography on silica gel, C8 or C18; preparative HPLC on silica gel columns, C8 or C18; and supercritical CO2 chromatography on silica gel.

Centrifugation partitioning chromatography (CPC) and counter current chromatography (CCC) can be used, e.g., in the extraction and enrichment of compounds from plant extracts in analytical, semi-preparative and preparative scale. CPC and CCC are a liquid-liquid chromatography methods using a mostly two-phase solvent. It enables an almost loss-free separation of complex mixtures of substances from crude extracts. CPC and CCC are comparable to liquid chromatography (HPLC) which can also be used according to the present teachings.

Mass spectrometry for quantitative analysis of the profile.

Specific conditions for HPLC are described below in the Examples section which follows.

Also provided is a method of producing cannabinoids in a plant part thereof or a cell as described herein, followed by recovering the cannabinoids such as described hereinabove and in the Examples section which follows.

Optionally, the process involves extraction and/or fractionation using methods which are well known in the art and described for example in U.S. Publ. Nos. 20190134532, 20180292369, 20190214145, 20190201809, and 20180222879, each of which is incorporated herein by reference in its entirety.

According to a specific embodiment, the extraction can be effected by air dried Cannabis strains extracted in ethanol. Following extraction ethanol is evaporated under reduced pressure at about 38° C. using a rotary evaporator (Laborata 4000; Heidolph Instruments GmbH & Co. KG; Germany). The extracts are reconstituted into a vehicle solution consisting of 1:1:18 ethanol:cremophor (Sigma-Aldrich):saline to a final concentration of 20 mg/ml.

The Cannabis extract can be injected and measured by HPLC.

Alternatively or additionally the sample of the extract may be analyzed using LC/MS by the described method for phytocannabinoid profiling.

Thus, the present procedures, plants, parts thereof and/or cells can yield a cannabinoid extract/preparation which was not described to date and can be used in various applications including medicinal and recreational.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Three genomes were download from NCBI: GCA_003417725.2_ASM341772v2 Finola, GCA_000230575.3_ASM23057v3 Purple Kush, GCA_003660325.2 Jamaican Lion DASH. Blastn against the 3 genomes evalue (<0.005)& identity (85%>).

In house editing the blast result for finding the non-redundant gene intervals.

Transcriptome Comparison

The candidates genes were compared (homology searches) to the expression profiles of 40226 PK accession transcripts from www(dot)ncbi(dot)nlm(dot)nih(dot)gov/geo/query/acc(dot)cgi?acc=GSE93201.

Phylogenetic Analysis

Ninety sequences were aligned using the MAFFT program (Version 7, www(dot)mafft(dot)cbrc(dot)jp/alignment/server/) with default parameters. Gblocks server was used for the selection of conserved blocks in the multiple alignment (Talavera, G., and Castresana, J. (2007). Improvement of phylogenies after removing divergent and ambiguously aligned blocks from protein sequence alignments. Systematic Biology 56, 564-577). A maximum likelihood tree with 100 bootstrap replicates using PhyML 3.0 software (Guindon et al., 2010) was constructed based on the automatic nucleotide model selection by AIC (Akaike Information Criterion) (“SMS: Smart Model Selection in PhyML.” Vincent Lefort, Jean-Emmanuel Longueville, Olivier Gascuel. Molecular Biology and Evolution, msx149, 2017). The tree was graphically designed using FigTree Version 1.4.2 (www(dot)tree(dot)bio(dot)ed(dot)ac(dot)uk/software/figtree/)

Gene Expression Analysis

Gene expression profiles from cannabis plant tissue at different developmental stages were downloaded from the NCBI GEO repository (www(dot)ncbi(dot)nlm(dot)nih(dot)gov/geo/). Gene expression heatmaps and unsupervised hierarchical clustering were performed with GENE-E 3.0.21329. The tissue profile was compered for PK and FN transcriptome data.

Promoter Analysis

In order to identify cis-regulatory elements within putative promoter regions; in silico analysis of the 5-UTRs (up to 1000 bp upstream of the putative translational start site) was conducted on 1K bp upstream of each sequence that was scanned for the presence of cis-acting regulatory elements involved in flowering gene expression pathway and plant hormones pathways based on the MatInspector program (www(dot)genomatix(dot)de).

### Results

Identification of Genes Associated with Cannabinoid Synthesis

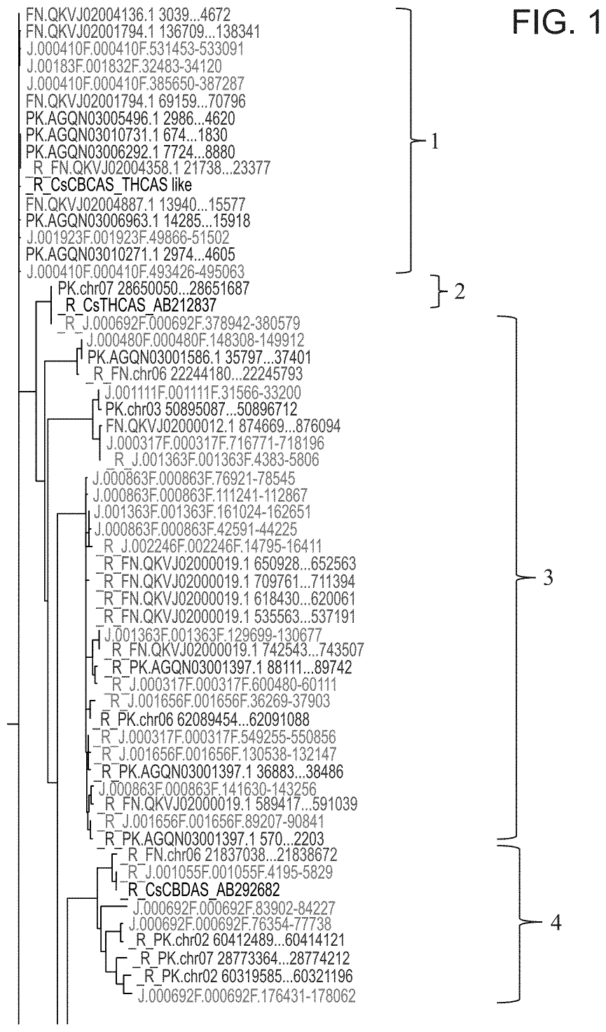

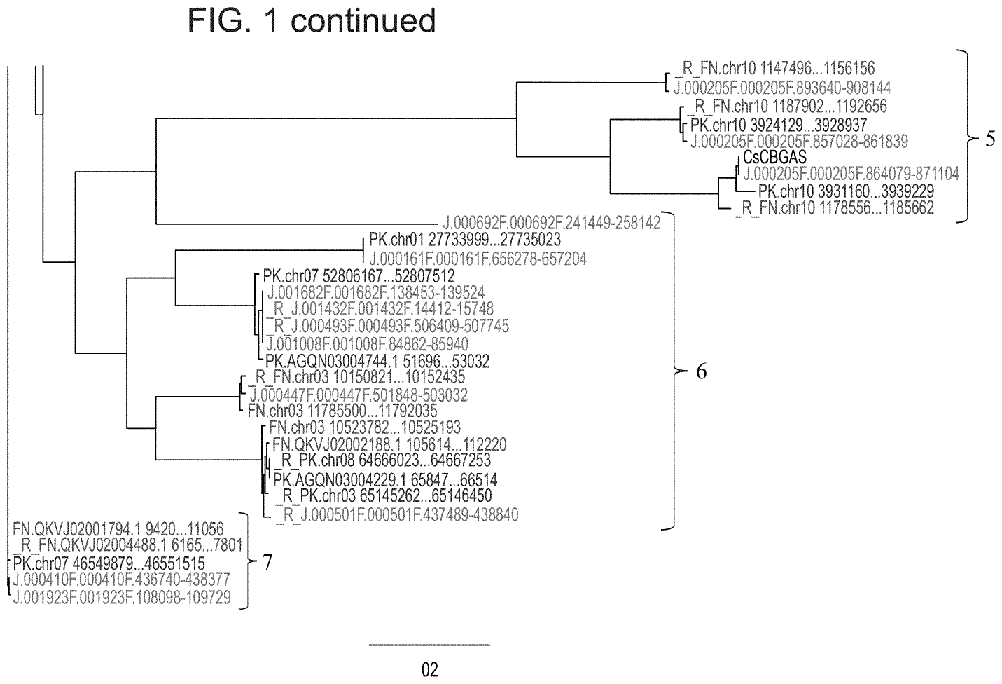

Three Cannabis sequenced genomes, JL-Jamaican lion, PK-purple kush and FN-finolla), were examined against four characterized cannabinoids synthases genes: THCAS (AB212837 in genebank), CBDAS (AB292682 in genebank), and GOT {olivetolate geranyltransferase, which together with the geranyl pyrophosphate (GPP) produce CBGAS (Cannabigerolic acid)}(BK010678.1 in genebank), and the cannabinoid synthase, CBCAS like (THCA2 or here defined as CBCAS like) (KJ469379.1 in genebank). These are termed reference sequences. The comparison, resulted in a polygenetic tree composed of: eight main groups, nineteen main branches, 84 different sequences located at the different genome loci (FIG. 1).

Gene Expression Profiles of the Genes

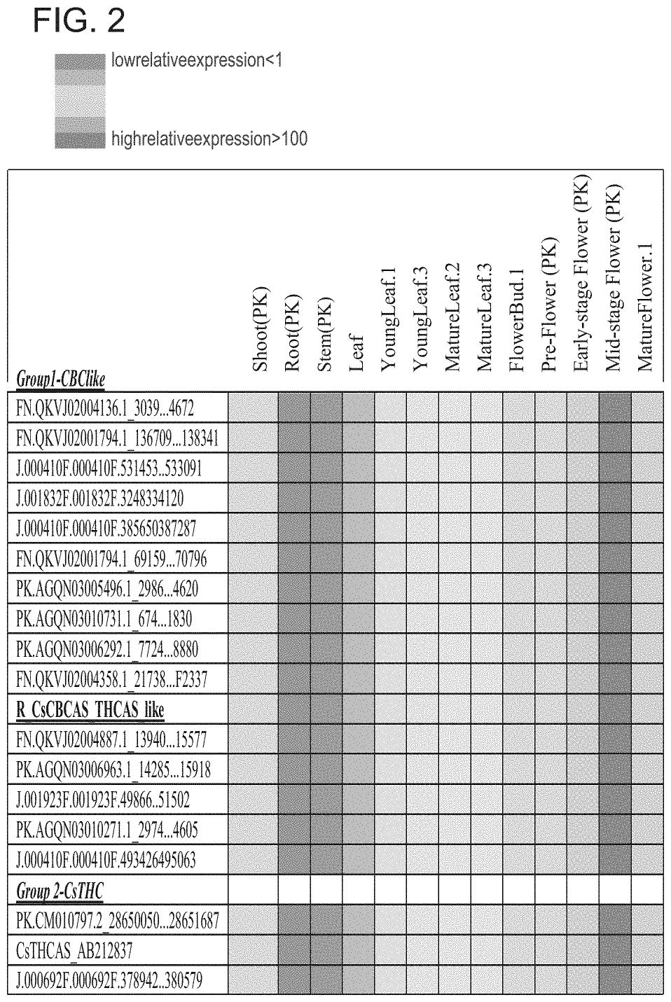

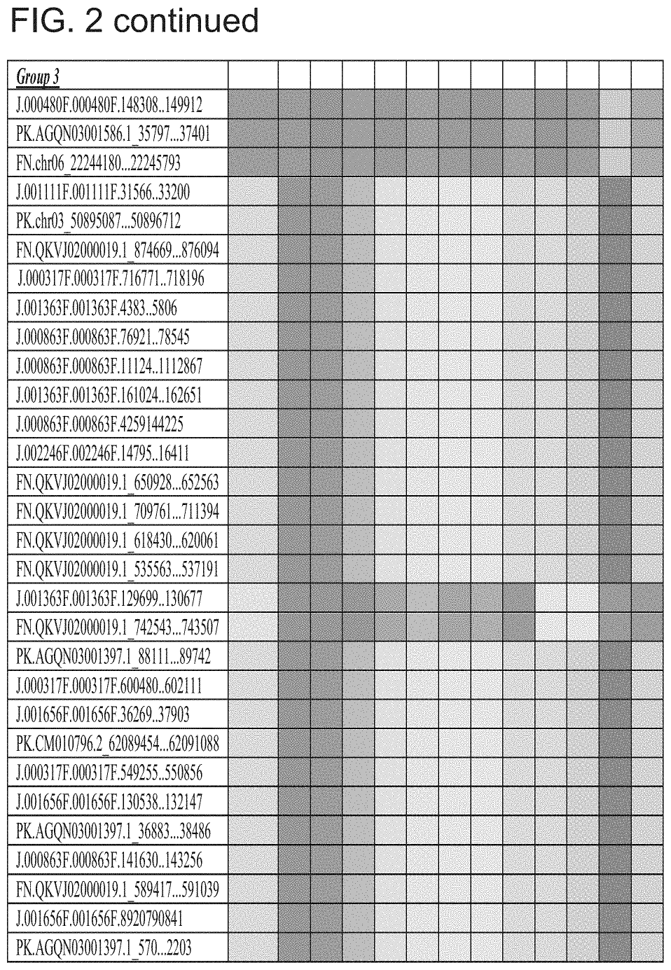

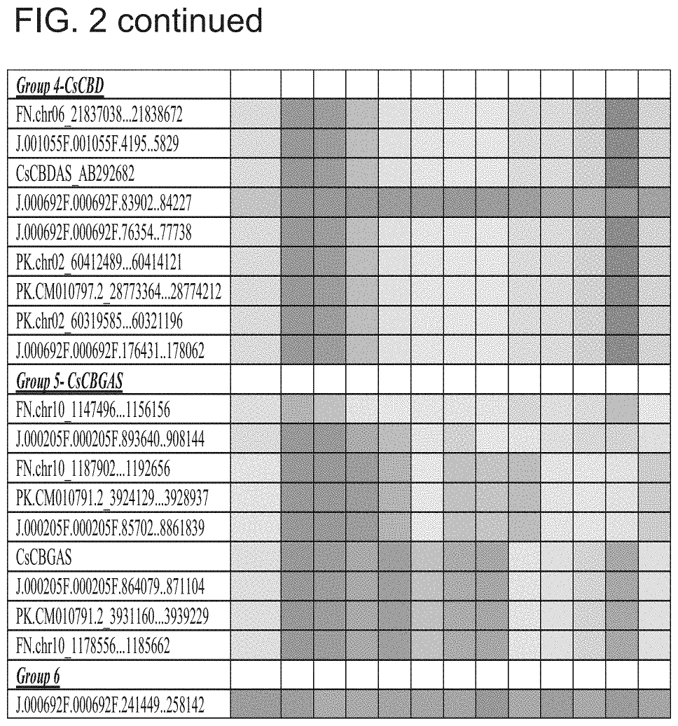

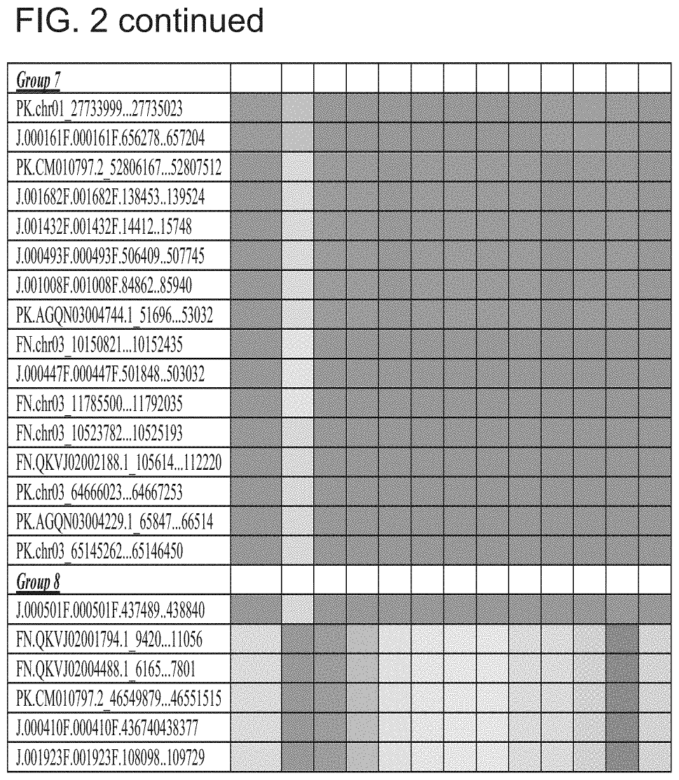

Gene expression profiles, of the 84 different genes, from cannabis plant tissue at different developmental stages were produced. Gene expression heatmaps and unsupervised hierarchical clustering were performed. In order to predict the involvement of the genes in phytocannabinoid metabolism, publicly-available (NCBI) gene expression data of FN and the PK genomes, in cannabis plant tissue at different developmental stages was used (FIG. 2).

Promoter Analysis

The DNA promoter region that initiates transcription of each gene was analyzed to identify the type of binding sites found in the region of the gene.

The upstream 1 K bp prior to the translational start sites of the genes were examined for the presence of various promoter elements. Elements common to 85% of the promoters and up, were examined (Table 1). Out of them 13 elements families, cohesive to flowering or plant hormones regulatory process were chosen. These families, as detailed in Table 1 below, were examined for each branch, seeking common regulatory in a uniformity regulatory elements binding sites sequences patterns.

TABLE 1
The common elements that were detected in the promoters regions of the Cannabis genes.

| Element | Response to | Reference |
|---|---|---|
| ARF3 | Auxin Response Factor 3 | Franco-Zorrilla J M, López-Vidriero I, Carrasco J L, Godoy M, Vera P, Solano R |
| | | DNA-binding specificities of plant transcription factors and |
| | | their potential to define target genes. |
| | | Proc Natl Acad Sci USA 111, 2367-72 (2014) |
| CE1F | a-biotic stress element | Molecular responses to dehydration and low temperature: |
| | | differences and cross-talk between two stress signaling pathways. |
| | | Shinozaki K, Yamaguchi-Shinozaki K |
| | | Curr Opin Plant Biol. 2000 Jun; 3(3):217-23 |
| CCAF | Circadian clock associated | A Myb-related transcription factor is involved in the |
| | | phytochrome regulation of an Arabidopsis Lhcb gene. |
| | | Plant Cell 9, 491-507 (1997) |
| | | DNA-binding specificities of plant transcription factors and |
| | | their potential to define target genes. |
| | | Proc Natl Acad Sci USA 111, 2367-72 (2014) |
| DREB | a-biotic stress element | Molecular responses to dehydration and low temperature: |
| | | differences and cross-talk between two stress signaling pathways. |
| | | Shinozaki K, Yamaguchi-Shinozaki K |
| | | Curr Opin Plant Biol. 2000 Jun; 3(3):217-23 |
| EINL | Ethylen insensitive 3 like | O'Malley R C, Huang S C, Song L, Lewsey M G, Bartlett A, |
| | factors | Nery J R, Galli M, Gallavotti A, Ecker J R |
| | | Cistrome and Epicistrome Features Shape the Regulatory DNA Landscape. |
| | | Cell 165, 1280-1292 (2016) |
| EREF | Ethylen response element | Nole-Wilson S, Krizek B A |
| | factors, | DNA binding properties of the Arabidopsis floral development |
| | AINTEGUMENTA in | protein AINTEGUMENTA. |
| | flowering response | Nucleic Acids Res 28, 4076-82 (2000) |
| GAPB | GAP-Box (light response | Cis-acting elements essential for light regulation of the nuclear |
| | elements) | gene encoding the A subunit of chloroplast glyceraldehyde 3- |
| | | phosphate dehydrogenase in Arabidopsis thaliana. |
| | | Plant Physiol 112, 1563-71 (1996) |
| | | Identification of a light-responsive region of the nuclear gene |
| | | encoding the B subunit of chloroplast glyceraldehyde 3- |
| | | phosphate dehydrogenase from Arabidopsis thaliana. |
| | | Plant Physiol 105, 357-67 (1994) |
| HEAT | Heat shock factors | Interaction between Arabidopsis heat shock transcription |
| | | factor 1 and 70 kDa heat shock proteins. J Exp Bot 53, 371-5 (2002) |
| | | Selective activation of the developmentally regulated Hahsp17.6 |
| | | G1 promoter by heat stress transcription factors. Plant |
| | | Physiol 129, 1207-15 (2002) |
| IBOX | light regulation | Interaction of a GATA factor with cis-acting elements |
| | | involved in light regulation of nuclear genes encoding |
| | | chloroplast glyceraldehyde-3-phosphate dehydrogenase in |
| | | Arabidopsis. Biochem Biophys Res Commun 300, 555-62 (2003) |
| | | Arabidopsis thaliana GATA factors: organization, expression |
| | | and DNA-binding characteristics. |
| | | Plant Mol Biol 50, 43-57 (2002) |
| JARE | Jasmonate response | The Arabidopsis JAZ2 promoter contains a G-Box and |
| | element | thymidine-rich module that are necessary and sufficient for |
| | | jasmonate-dependent activation by MYC transcription factors |
| | | and repression by JAZ proteins. Plant Cell Physiol 53, 330-43 (2012) |
| LREM | Light responsive element | Welsch R, Maass D, Voegel T, Dellapenna D, Beyer P. |
| | motif | Transcription factor RAP2.2 and its interacting partner |
| | | SINAT2: stable elements in the carotenogenesis of Arabidopsis leaves. |
| | | Plant Physiol 145, 1073-85 (2007) |
| STKM | Storekeeper motif | Storekeeper defines a new class of plant-specific DNA-binding |
| | | proteins and is a putative regulator of patatin expression. Plant |
| | | J 30, 489-97 (2002) |
| TOEF | Target of early activation | DNA-binding specificities of plant transcription factors and their |
| | tagged factors -AP2 domain | potential to define target genes. Proc Natl Acad Sci USA 111, 2367-72 (2014) |

Sequences Analysis of the Promoter Region and Expression Profile

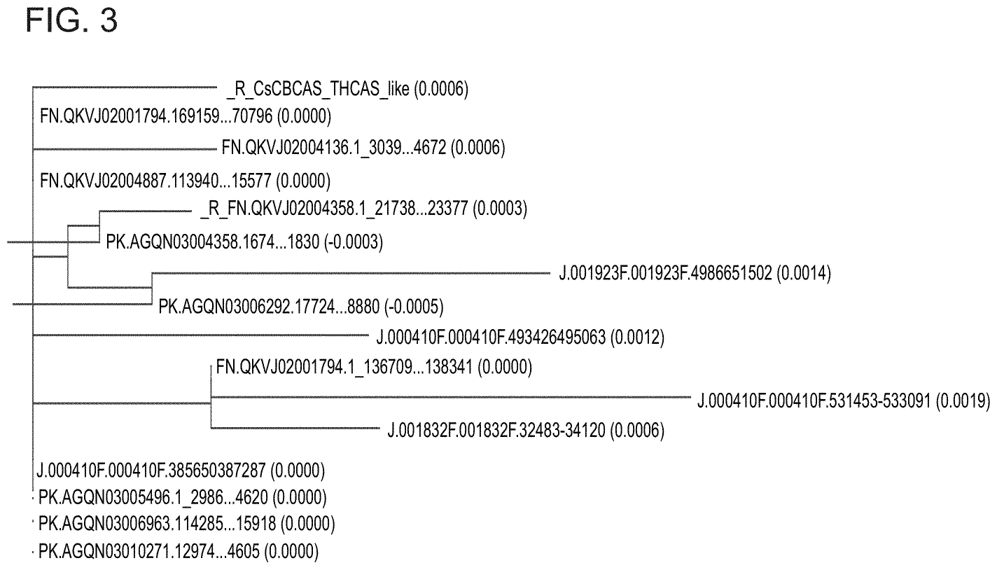

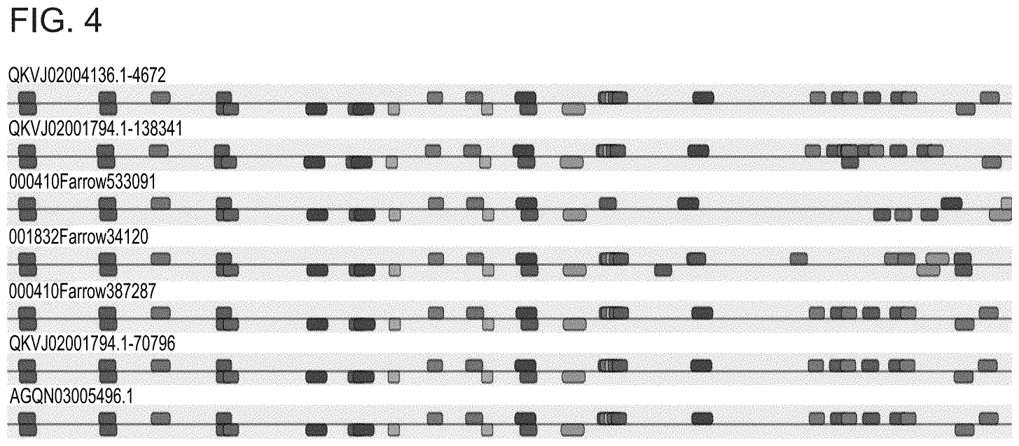

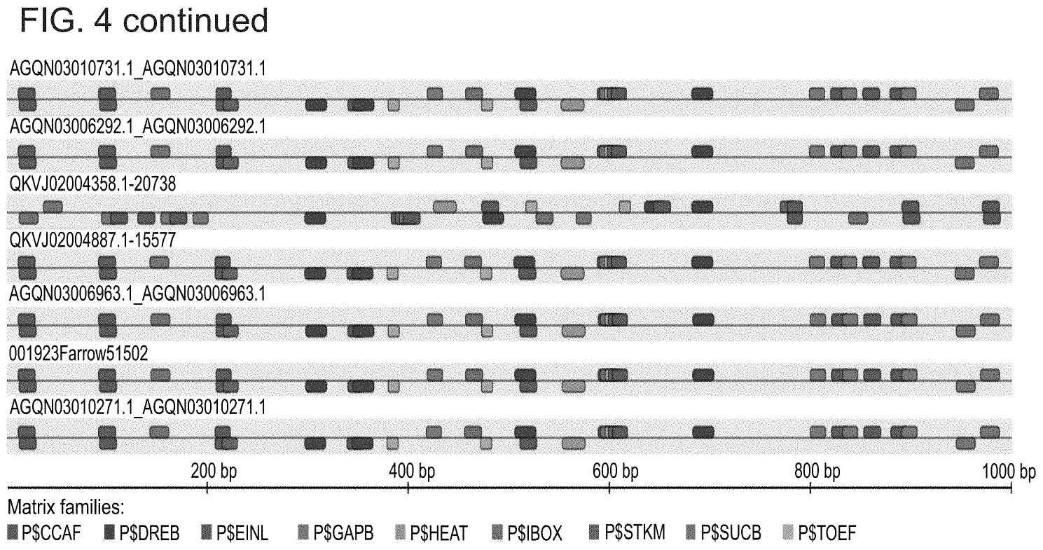

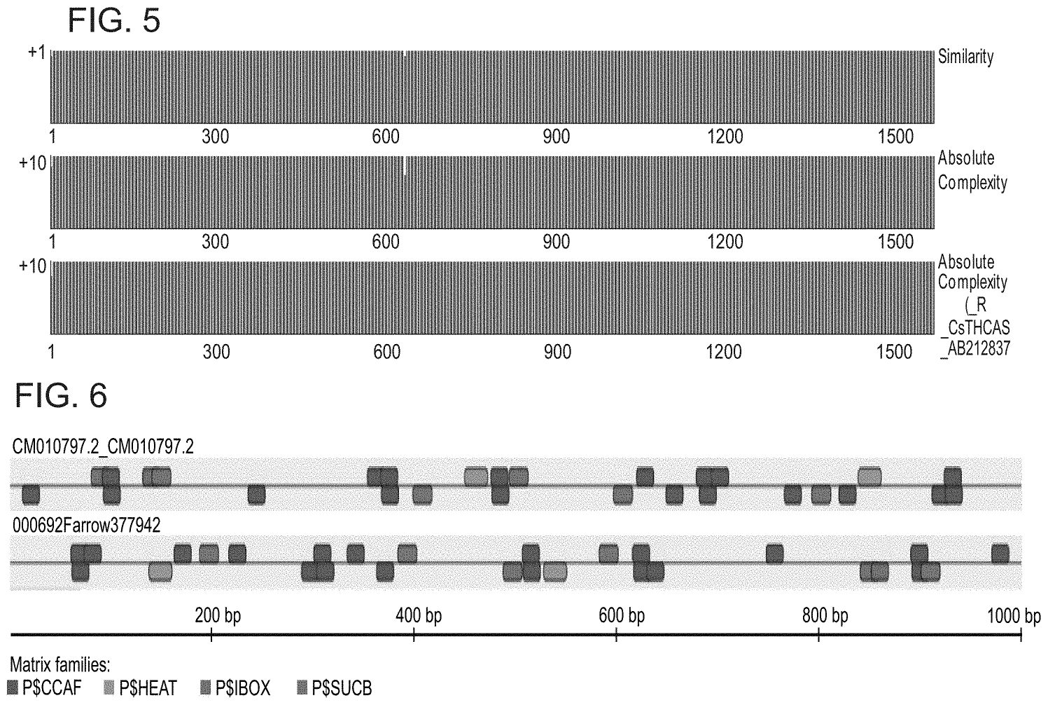

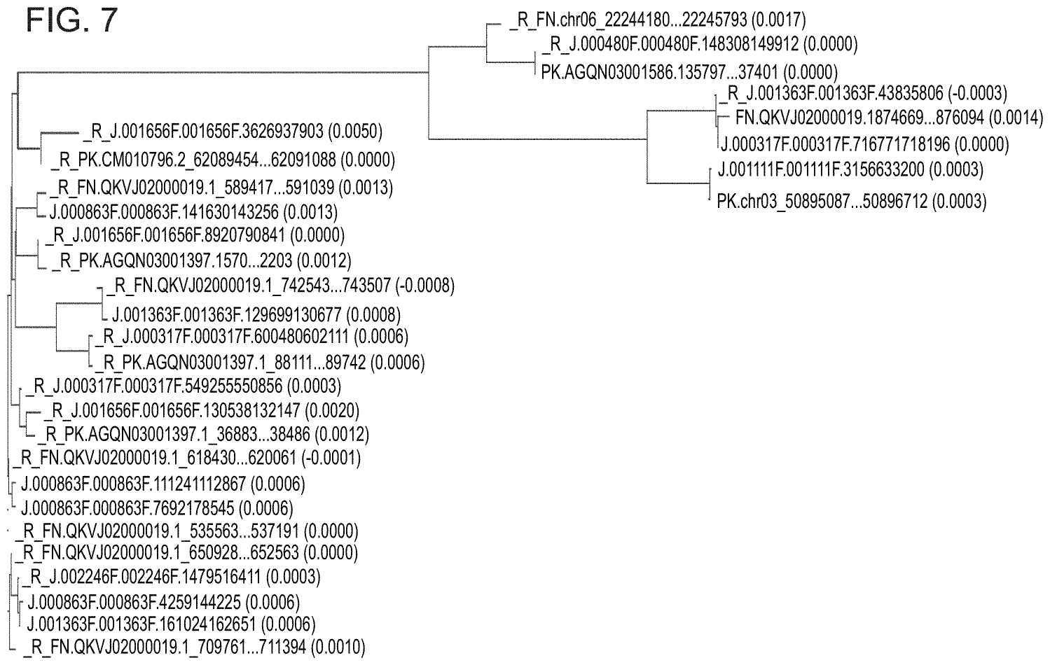

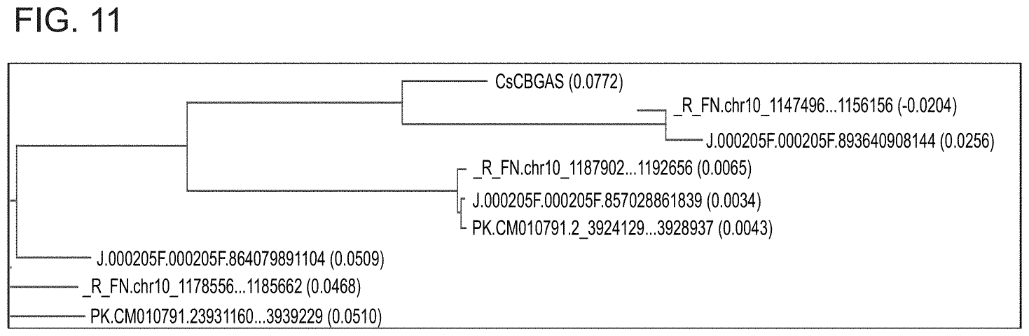

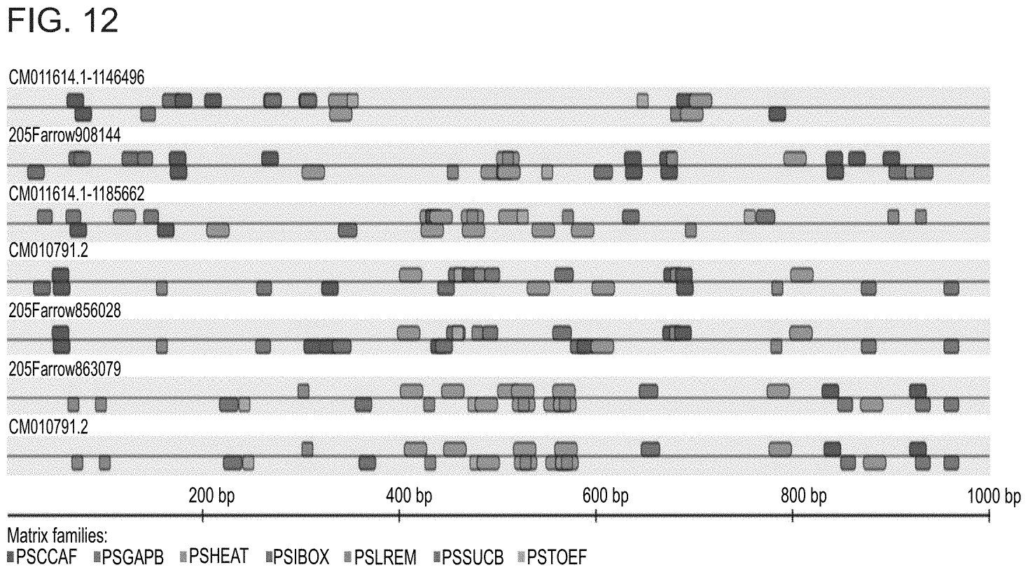

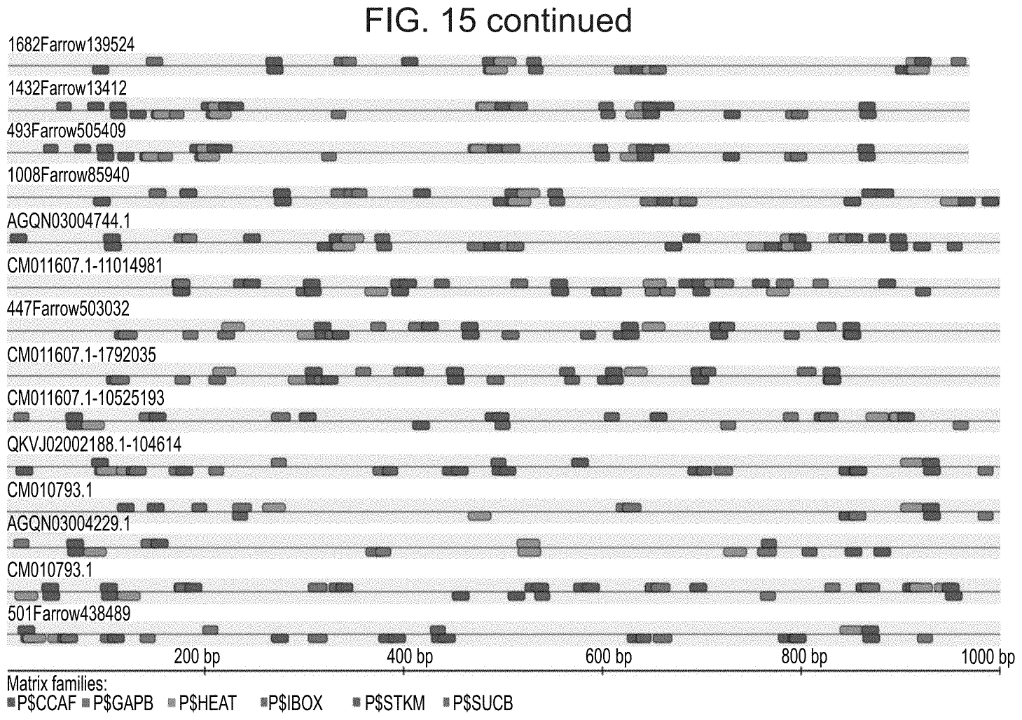

The following data shows the sequences of each gene and promoter region divided by the eight main groups created by the phylogenetic analysis (FIGS. 3-19A-E).

### Example 1.1

### Group 1 (CBCAS Like Genes)

This first branch is composed of 15 genes. CBCAS like genes (FIG. 3) were found in all the 3 genomes in 5 copies (indicating amplification), exhibiting 99% nucleic acid sequence identity. The sequences are highly conserved and their promotor region shows similar controlling groups. The promotor conservation was found in the two examined genomes of FN (representing hemp group) and PK (representing medicinal (drug)-type strains). Differences were found in the expression pattern of the genes in the two genomes: in PK high expression was found in the flower and the vegetative part while in FN high expression is was found in the seeds and young flowers. However, on both flower expression was evident in different development stages.

TABLE 2

| | | Chromosome | | Number | | |
|---|---|---|---|---|---|---|
| | Genome JL- | orientation | | of genes | | |
| | Jamaican lion | no chromosome | | and copies | Tissue | Tissue |
| Group 1-CsCBCAS like/ | PK-purple Kush | orientations, | | 1 gene | associated | associated |
| SEQ ID NO: 87-101 | FN-finolla | PK chr07 | Exons | 5 copies | by PK | by FN |
| PK.AGQN03010731.1 | PK | | no | | shoot and | seed and |
| 674 . . . 1830 | | | introns | | flower | mid flower |
| PK.AGQN03006963.1 | PK | | no | | shoot and | seed and |
| 14285 . . . 15918 | | | introns | | flower | mid flower |
| PK.AGQN03006292.1 | PK | | no | | shoot and | seed and |
| 7724 . . . 8880 | | | introns | | flower | mid flower |
| FN.QKVJ02004136.1 | FN | | no | | shoot and | seed and |
| 3039 . . . 4672 | | | introns | | flower | mid flower |
| PK.AGQN03005496.1 | PK | | no | | shoot and | seed and |
| 2986 . . . 4620 | | | introns | | flower | mid flower |
| CsCBCAS KJ469379.1 | | | | | | |
| PK.AGQN03010271.1 | PK | | no | | shoot and | seed and |
| 2974 . . . 4605 | | | introns | | flower | mid flower |
| J.001923F.001923F.49866- | JL | | no | | shoot and | seed and |
| 51502 | | | introns | | flower | mid flower |
| FN.QKVJ02001794.1 | FN | | no | | shoot and | seed and |
| 69159 . . . 70796 | | | introns | | flower | mid flower |
| J.000410F.000410F.385650- | JL | | no | | shoot and | seed and |
| 387287 | | | introns | | flower | mid flower |
| _R_FN.QKVJ02004358.1 | FN | | no | | shoot and | seed and |
| 21738 . . . 23377 | | | introns | | flower | mid flower |
| J.000410F.000410F.531453- | JL | | no | | shoot and | seed and |
| 533091 | | | introns | | flower | mid flower |
| J.001832F.001832F.32483- | JL | | no | | shoot and | seed and |
| 34120 | | | introns | | flower | mid flower |
| FN.QKVJ02001794.1 | FN | | no | | shoot and | seed and |
| 136709 . . . 138341 | | | introns | | flower | mid flower |
| FN.QKVJ02004887.1 | FN | | no | | shoot and | seed and |
| 13940 . . . 15577 | | | introns | | flower | mid flower |
| J.000410F.000410F.493426- | JL | PK-chr7 | no | | shoot and | seed and |
| 495063 | | | introns | | flower | mid flower |

### Example 1.2

### Group 3

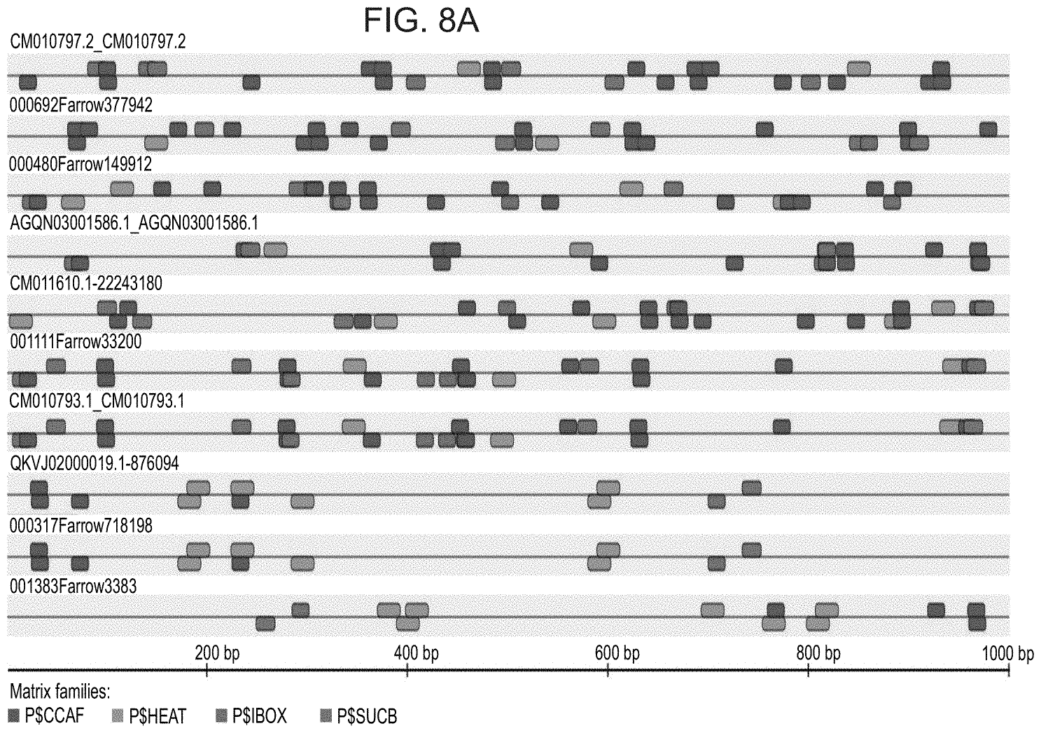

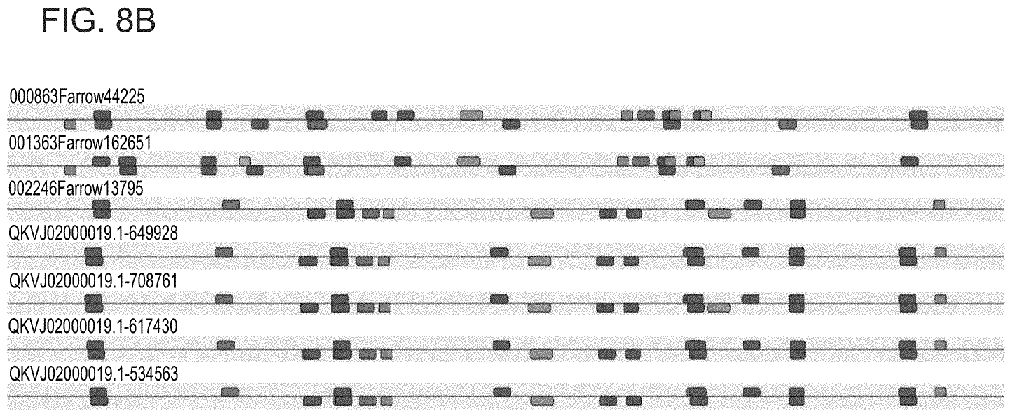

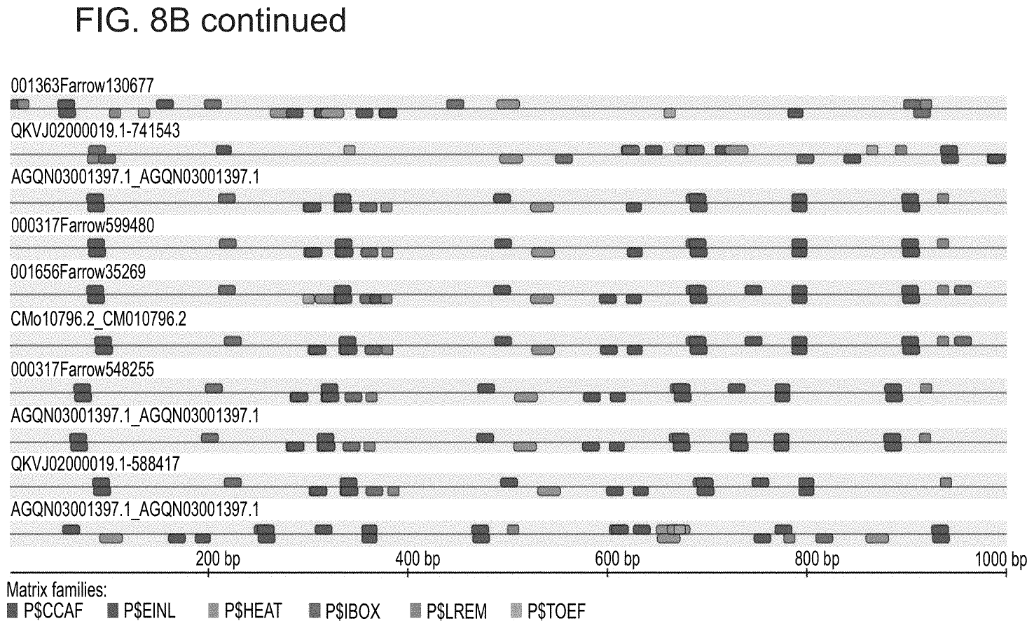

Group 3 is the largest identified. The genes copies present at least 99% nucleic acid sequence identity. This branch is composed of 22 genes. The controlling elements vary in this group (FIGS. 8A-B). The expression of the genes is different between the FN and PK (Table 3), although both present flower expression.

TABLE 3

| | Genome JL- | | | | | |
|---|---|---|---|---|---|---|
| | Jamaican lion | | | number | tissue | tissue |
| Group 3/SEQ ID | PK-purple Kush | Chromosome | | of genes | associated | associated |
| NO: 104-133 | FN-finolla | orientation | exons | and copies | by PK | by FN |
| FN.QKVJ02000019.1 | FN | | no | 13 genes | flower | low in mid flower |
| 709761 . . . 711394 | | | introns | | and shoot | male flower and seed |
| J.002246F.002246F.14795- | JL | | no | | flower | low in mid flower |
| 16411 | | | introns | | and shoot | male flower and seed |
| J.000863F.000863F.42591- | JL | | no | | flower | low in mid flower |
| 44225 | | | introns | | and shoot | male flower and seed |
| J.001363F.001363F.161024- | JL | | no | | flower | low in mid flower |
| 162651 | | | introns | | and shoot | male flower and seed |
| FN.QKVJ02000019.1 | FN | | no | | flower | low in mid flower |
| 650928 . . . 652563 | | | introns | | and shoot | male flower and seed |
| FN.QKVJ02000019.1 | FN | | no | | flower | low in mid flower |
| 618430 . . . 620061 | | | introns | | and shoot | male flower and seed |
| FN.QKVJ02000019.1 | FN | | no | | flower | low in mid flower |
| 535563 . . . 537191 | | | introns | | and shoot | male flower and seed |
| J.001656F.001656F.89207- | JL | | no | | flower | low in mid flower |
| 90841 | | | introns | | and shoot | male flower and seed |
| PK.AGQN03001397.1 | PK | | no | | flower | low in mid flower |
| 570 . . . 2203 | | | introns | | and shoot | male flower and seed |
| FN.QKVJ02000019.1 | FN | | no | | flower | low in mid flower |
| 589417 . . . 591039 | | | introns | | and shoot | male flower and seed |
| J.000863F.000863F.141630- | JL | PK-chr7 | no | | flower | low in mid flower |
| 143256:0.001201 | | | introns | | and shoot | male flower and seed |
| J.001656F.001656F.130538- | JL | PK-chr6 | no | | flower | low in mid flower |
| 132147 | | | introns | | and shoot | male flower and seed |
| PK.AGQN03001397.1 | PK | | no | | flower | low in mid flower |
| 36883 . . . 38486 | | | introns | | and shoot | male flower and seed |
| J.000317F.000317F.549255- | JL | | no | | flower | low in mid flower |
| 550856 | | | introns | | and shoot | male flower and seed |
| FN.QKVJ02000019.1 | FN | | no | | flower | low in mid flower |
| 742543 . . . 743507 | | | introns | | and shoot | male flower and seed |
| J.001363F.001363F.129699- | JL | | no | | flower | low in mid flower |
| 130677 | | | introns | | and shoot | male flower and seed |
| PK.AGQN03001397.1 | PK | | no | | flower | low in mid flower |
| 88111 . . . 89742 | | | introns | | and shoot | male flower and seed |
| J.000317F.000317F.600480- | JL | | no | | flower | low in mid flower |
| 602111 | | | introns | | and shoot | male flower and seed |
| PK.chr06 | PK | | no | | flower | low in mid flower |
| 62089454 . . . 62091088 | | | introns | | and shoot | male flower and seed |
| J.001656F.001656F.36269- | JL | | no | | flower | low in mid flower |
| 37903 | | | introns | | and shoot | male flower and seed |
| J.000863F.000863F.76921- | JL | | no | | flower | low in mid flower |
| 78545 | | | introns | | and shoot | male flower and seed |
| J.000863F.000863F.111241- | JL | | no | | flower | low in mid flower |
| 112867 | | | introns | | and shoot | male flower and seed |

### Example 1.3

### Group 4 (CBDAS Like Genes)

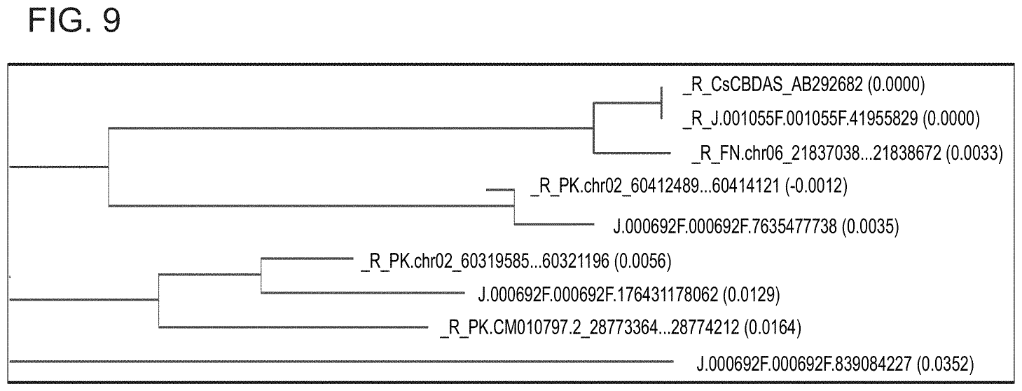

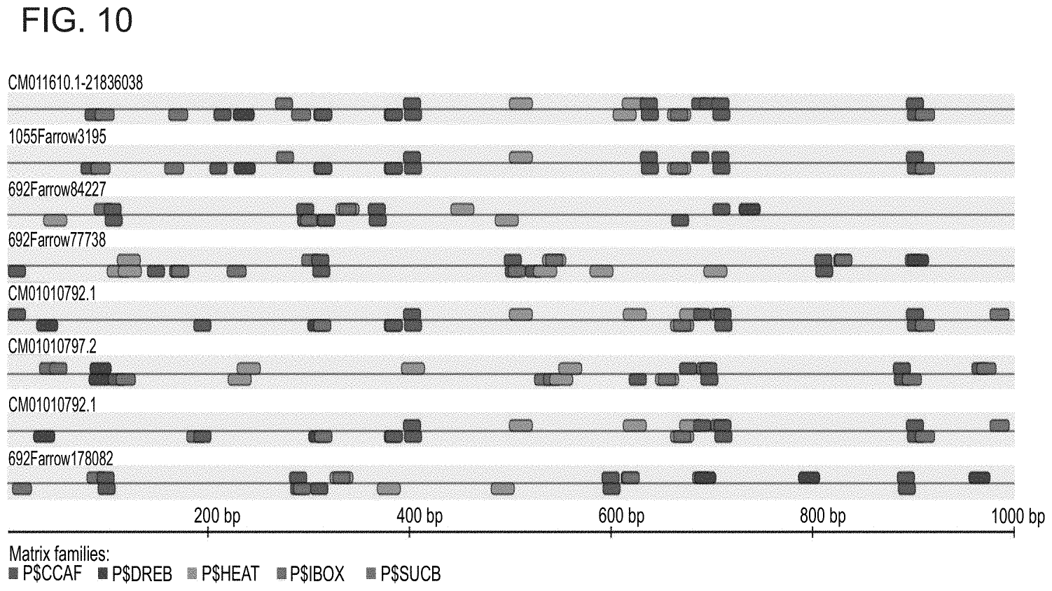

The forth branch is of the CBDAS like genes, composed of 8 genes in the three genomes. These genes exhibited at least 99% nucleic acid sequence identity (FIG. 9). Diverse motifs were found in the promoter regions of the genes (FIG. 10) and differences in the expression tissues (Table 4) albeit flower expression was evident in all.

TABLE 4

| | | | | | | |
|---|---|---|---|---|---|---|
| | | | | number | | |
| | Genome JL- | Chromosome | | of genes | | |
| | Jamaican lion | orientation | | and copies | tissue | tissue |
| Group 4- CsCBD/SEQ | PK-purple Kush | PK-chr2, chr7 | | up to | associated | associated |
| ID NO: 134-141 | FN-finolla | PN-chr6 | exons | 6 genes | by PK | by FN |

| | | | | |
|---|---|---|---|---|
| PK.chr02 | PK | no | low in flower | very low in mid |
| 60319585 . . . 60321196 | | introns | and shoot | flower and seed |
| J.000692F.000692F.176431- | JL | no | low in flower | very low in mid |
| 178062 | | introns | and shoot | flower and seed |
| PK.chr07 | PK | no | low in flower | very low in mid |
| 28773364 . . . 28774212 | | introns | and shoot | flower and seed |
| PK.chr02 | PK | no | low in flower | very low in mid |
| 60412489 . . . 60414121 | | introns | and shoot | flower and seed |
| J.000692F.000692F.76354- | JL | no | low in flower | very low in mid |
| 77738 | | introns | and shoot | flower and seed |
| J.000692F.000692F.83902- | JL | no | low in flower | very low in mid |
| 84227 | | introns | and shoot | flower and seed |
| CsCBDAS AB292682 | | no | low in flower | very low in mid |
| | | introns | and shoot | flower and seed |
| J.001055F.001055F.4195- | JL | no | low in flower | very low in mid |
| 5829 | | introns | and shoot | flower and seed |
| FN.chr06 | FN | no | low in flower | very low in mid |
| 21837038 . . . 21838672 | | introns | and shoot | flower and seed |

### Example 1.4

### Group 5 (CBGAS Like Genes)

The CBGAS like branch is composed of eight genes. The CBGAS is the only one assembly with 10 exons, in the three homologs. The homologs exhibit more than 99% nucleic acid sequence identity and all mapped in FN and PK genomes to chromosome 10. Minor expression is evident in all plant tissues, except to flower tissue where it increases dramatically. Promoter analysis showed binding elements involved in the flowering progress: (TOEF), light response cascades (IBOX, GAPB), circadian clock cascade (CCAF) and heat and Jasmonate as stress recons (JARE, HEAT).

TABLE 5

| | | | | | | |
|---|---|---|---|---|---|---|
| | | | | number | | |
| | Genom JL- | Chromozom | | of genes | | |
| | jamaican lion | oraintation | | and copies | tissue | tissue |
| Group 5- CBGAS/SEQ | PK-purpel Kush | PK-chr10 | | up to | associated | associated |
| ID NO: 142-149 | FN-finolla | FN-chr10 | exons | 7 genes | by PK | by FN |

| | | | | |
|---|---|---|---|---|
| J.000205F.000205F.864079- | JL | 10 | very low in | very low in mid |
| 871104 | | | all parts | flower and seed |
| PK.chr10 | PK | 10 | flower | very low in mid |
| 3931160 . . . 3939229 | | | and shoot | flower and seed |
| _R_FN.chr10 | FN | 10 | flower | very low in mid |
| 1178556 . . . 1185662 | | | and shoot | flower and seed |
| PK.chr10 | PK | 10 | undetected | undetected |
| 3924129 . . . 3928937 | | | | |
| J.000205F.000205F.857028- | JL | 10 | undetected | undetected |
| 861839 | | | | |
| _R_FN.chr10 | FN | 10 | undetected | undetected |
| 1187902 . . . 1192656 | | | | |
| _R_FN.chr10 | FN | 10 | flower | low in mid an male |
| 1147496 . . . 1156156 | | | and shoot | flower and seed |
| J.000205F.000205F.893640- | JL | 10 | flower | low in mid an male |
| 908144 | | | and shoot | flower and seed |

### Example 1.5

### Group 6

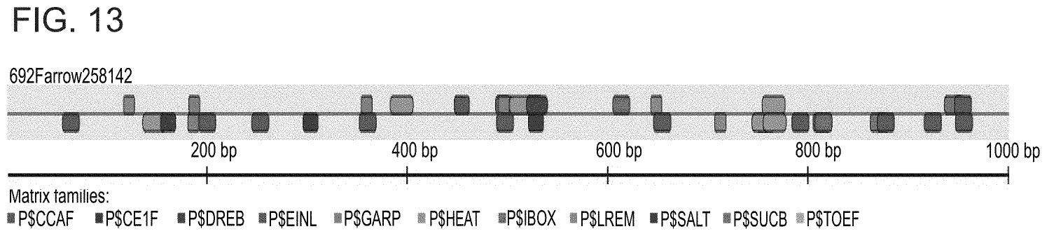

This branch was found in JL genome. It has one sequence with 10 exons and promoter elements (FIG. 13).

TABLE 6

| | Genome JL- | | | | | |
|---|---|---|---|---|---|---|
| | Jamaican lion | | | number | tissue | tissue |
| Group 6/SEQ | PK-purple Kush | Chromosome | | of genes | associated | associated |
| ID NO: 150 | FN-finolla | orientation | exons | and copies | by PK | by FN |
| J.000692F.000692F.241449- | JL | No | 10 | 1 gene | undetected | undetected |
| 258142 | | chromozom | | | | |

### Example 1.6

### Group 7

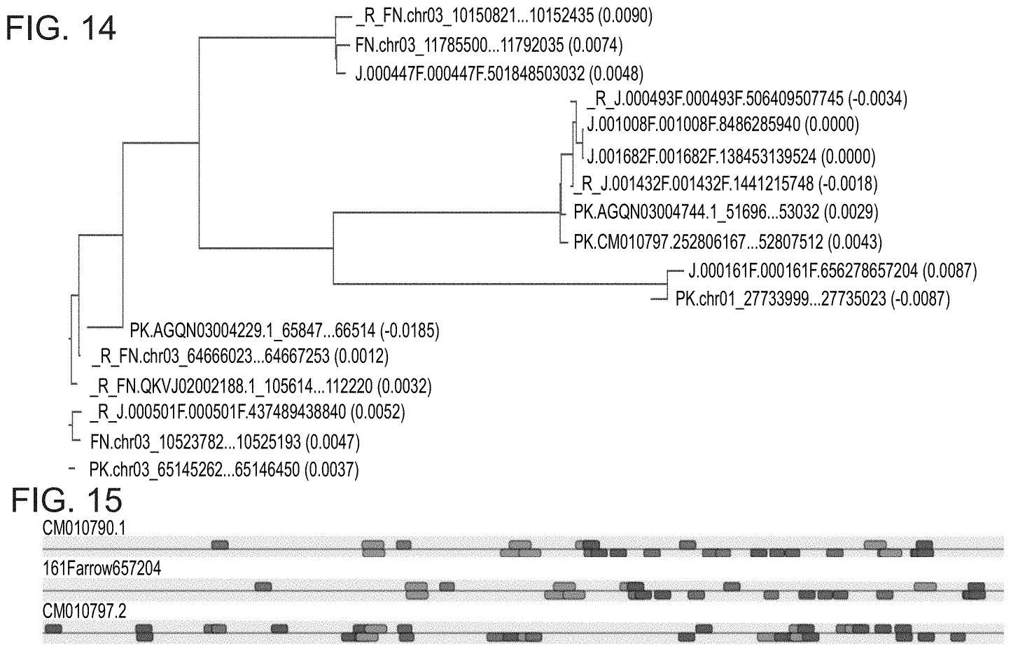

Composed of 25 genes. The phylogenetic analysis shows up to 7 main candidate genes, exhibiting at least 99% nucleic acid sequence identity (FIG. 14). Diversity in the controlling elements and expression pattern is shown in Table 7.

TABLE 7

| | Genome JL- | | | | | |
|---|---|---|---|---|---|---|
| | Jamaican lion | | | number | tissue | tissue |
| Group 7/SEQ | PK-purple Kush | Chromosome | | of genes | associated | associated |
| ID NO: 151-167 | FN-finolla | orientation | exons | and copies | by PK | by FN |
| _R_J.000493F.000493F.506409- | JL | PK-chr7 | no | up to | ROOT | undetected |
| 507745 | | | introns | 11 genes | | |
| _R_J.001432F.001432F. 14412- | JL | | no | | ROOT | undetected |
| 15748 | | | introns | | | |
| J.001682F.001682F.138453- | JL | PK-chr7 | no | | ROOT | undetected |
| 139524 | | | introns | | | |
| PK.AGQN03004744.1 | PK | | no | | ROOT | undetected |
| 51696 . . . 53032 | | | introns | | | |
| J.001008F.001008F.84862- | JL | | no | | ROOT | undetected |
| 85940 | | | introns | | | |
| PK.chr07 | PK | | no | | ROOT | undetected |
| 52806167 . . . 52807512 | | | introns | | | |
| PK.chr01 | PK | | no | | very low | undetected |
| 27733999 . . . 27735023 | | | introns | | in root | |
| J.000161F.000161F.656278- | JL | PK-chr1 | no | | very low | undetected |
| 657204 | | | introns | | in root | |
| J.000447F.000447F.501848- | JL | FN-chr3 | no | | ROOT | undetected |
| 503032 | | PK-chr3 | introns | | | |
| _R_FN.chr03 | FN | | no | | root | undetected |
| 10150821 . . . 10152435 | | | introns | | | |
| FN.chr03 | FN | | no | | root | undetected |
| 11785500 . . . 11792035 | | | introns | | | |
| FN.chr03 | FN | | no | | root | undetected |
| 10523782 . . . 10525193 | | | introns | | | |
| _R_FN.QKVJ02002188.1 | FN | | no | | root | undetected |
| 105614 . . . 112220 | | | introns | | | |
| _R_PK.chr03 | PK | | no | | root | undetected |
| 64666023 . . . 64667253 | | | introns | | | |
| PK.AGQN03004229.1 | PK | | no | | root | seed and |
| 65847 . . . 66514 | | | introns | | | mid flower |
| PK.chr03 | PK | | no | | root | undetected |
| 65145262 . . . 65146450 | | | introns | | | |
| _R_J.000501F.000501F.437489- | JL | | no | | root | undetected |
| 438840 | | | introns | | | |
| J.000317F.000317F.716771- | JL | | no | | shoot and | seed and |
| 718196 | | | introns | | flower | mid flower |
| _R_J.001363F.001363F.4383- | JL | | no | | shoot and | seed and |
| 5806 | | | introns | | flower | mid flower |
| FN.QKVJ02000019.1 | FN | | no | | shoot and | seed and |
| 874669 . . . 876094 | | | introns | | flower | mid flower |
| J.001111F.001111F.31566- | JL | PK-chr3 | no | | shoot and | seed and |
| 33200 | | | introns | | flower | mid flower |
| PK.chr03 | FN | | no | | shoot and | seed and |
| 50895087 . . . 50896712 | | | introns | | flower | mid flower |
| _R_FN.chr06 | FN | | no | | shoot and | seed and |
| 22244180 . . . 22245793 | | | introns | | flower | mid flower |
| PK.AGQN03001586.1 | PK | | no | | shoot and | seed and |
| 35797 . . . 37401 | | | introns | | flower | mid flower |
| J.000480F.000480F.148308- | JL | PN-chr6 | no | | shoot and | seed and |
| 149912 | | | introns | | flower | mid flower |

### Example 1.7

### Group 8

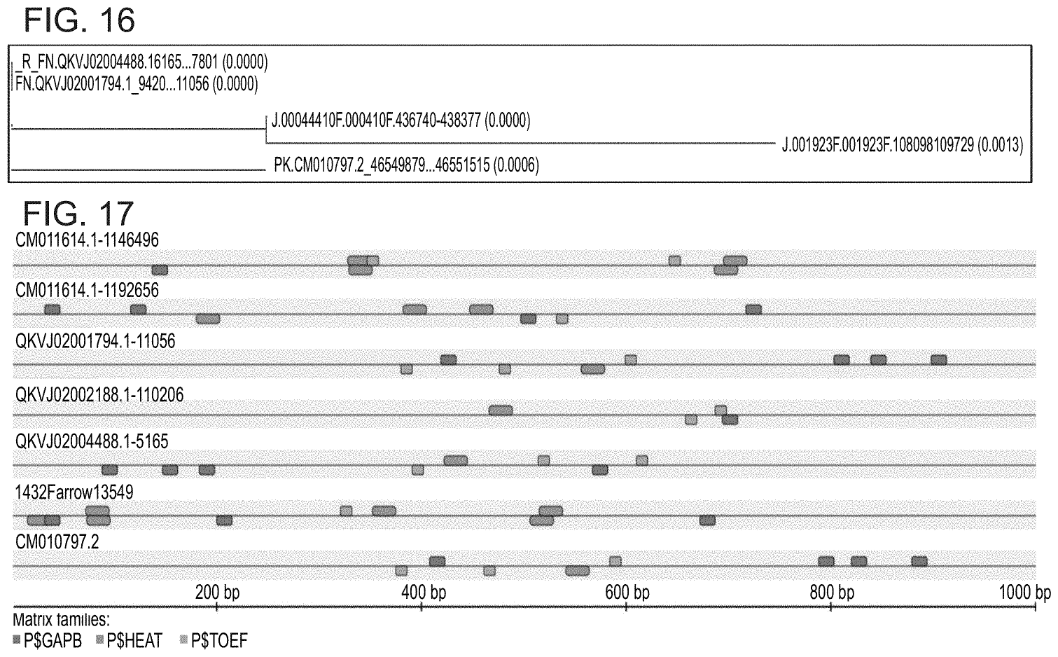

Composed of 5 genes. This branch is shown in JL and FN in two copies and in PK in one. They exhibit at least 99% identity (FIG. 16). However, the controlling elements are highly variable (FIG. 17).

TABLE 8

| | | | | number | | |
|---|---|---|---|---|---|---|
| | Genome JL- | | | of genes | | |
| | Jamaican lion | | exons | and copies | tissue | tissue |
| Group 8/SEQ ID | PK-purple Kush | Chromosome | no | 1 gene | associated | associated |
| NO: 168-172 | FN-finolla | orientation | introns | 1-2 copies | by PK | by FN |
| PK.chr07 | PK | no | no | | shoot and | seed and |
| 46549879 . . . 46551515 | | | introns | | flower | mid flower |
| _R_FN.QKVJ02004488.1 | FN | | no | | shoot and | seed and |
| 6165 . . . 7801 | | | introns | | flower | mid flower |
| FN.QKVJ02001794.1 | FN | | no | | shoot and | seed and |
| 9420 . . . 11056 | | | introns | | flower | mid flower |
| J.000410F.000410F.436740- | JL | | no | | shoot and | seed and |
| 438377 | | | introns | | flower | mid flower |
| J.001923F.001923F.108098- | JL | | no | | shoot and | seed and |
| 109729 | | | introns | | flower | mid flower |

TABLE 9
Summary of all 90 candidates.

| | Genome JL- | | | | |
|---|---|---|---|---|---|
| | Jamaican lion | | | number | |
| | PK-purple Kush | Chromosome | | of genes | tissue |
| | FN-finolla | orientation | exons | and copies | associated |
| Group 5-CBGAS | | PK-chr10 | | up to 7 | |
| | | FN-chr10 | | genes | |
| J.000205F.000205F.864079- | JL | | 10 | | flower |
| 871104 | | | | | |
| PK.chr10 | PK | | 10 | | flower |
| 3931160 . . . 3939229 | | | | | |
| _R_FN.chr10 | FN | | 10 | | flower |
| 1178556 . . . 1185662 | | | | | |
| PK.chr10 | PK | | 10 | | flower |
| 3924129 . . . 3928937 | | | | | |
| J.000205F.000205F.857028- | JL | | 10 | | flower |
| 861839 | | | | | |
| _R_FN.chr10 | FN | | 10 | | flower |
| 1187902 . . . 1192656 | | | | | |
| _R_FN.chr10 | FN | | 10 | | flower |
| 1147496 . . . 1156156 | | | | | |
| J.000205F.000205F.893640- | JL | | 10 | | flower |
| 908144 | | | | | |
| Group 6 | | | | | |
| J.000692F.000692F.241449- | JL | | 10 | 1 gene | flower |
| 258142 | | | | | |
| Group 3 | | | | | |
| _R_FN.QKVJ02000019.1 | FN | | no | 13 genes | flower |
| 709761 . . . 711394 | | | introns | | |
| _R_J.002246F.002246F.14795- | JL | | no | | flower |
| 16411 | | | introns | | |
| J.000863F.000863F.42591- | JL | | no | | flower |
| 44225 | | | introns | | |
| J.001363F.001363F.161024- | JL | | no | | flower |
| 162651 | | | introns | | |
| _R_FN.QKVJ02000019.1 | FN | | no | | flower |
| 650928...652563 | | | introns | | |
| _R_FN.QKVJ02000019.1 | FN | | no | | flower |
| 618430...620061 | | | introns | | |
| _R_FN.QKVJ02000019.1 | FN | | no | | flower |
| 535563...537191 | | | introns | | |
| _R_J.001656F.001656F.89207- | JL | | no | | flower |
| 90841 | | | introns | | |
| _R_PK.AGQN03001397.1 | PK | | no | | flower |
| 570 . . . 2203 | | | introns | | |
| _R_FN.QKVJ02000019.1 | FN | | no | | flower |
| 589417 . . . 591039 | | | introns | | |
| J.000863F.000863F.141630- | JL | PK-chr7 | no | | flower |
| 143256:0.001201 | | | introns | | |
| _R_J.001656F.001656F.130538- | JL | PK-chr6 | no | | flower |
| 132147 | | | introns | | |
| _R_PK.AGQN03001397.1 | PK | | no | | flower |
| 36883 . . . 38486 | | | introns | | |
| _R_J.000317F.000317F.549255- | JL | | no | | flower |
| 550856 | | | introns | | |
| _R_FN.QKVJ02000019.1 | FN | | no | | flower |
| 742543 . . . 743507 | | | introns | | |
| J.001363F.001363F.129699- | JL | | no | | flower |
| 130677 | | | introns | | |
| _R_PK.AGQN03001397.1 | PK | | no | | flower |
| 88111 . . . 89742 | | | introns | | |
| _R_J.000317F.000317F.600480- | JL | | no | | flower |
| 602111 | | | introns | | |
| _R_PK.chr06 | PK | | no | | flower |
| 62089454 . . . 62091088 | | | introns | | |
| _R_J.001656F.001656F.36269- | JL | | no | | flower |
| 37903 | | | introns | | |
| J.000863F.000863F.76921- | JL | | no | | flower |
| 78545 | | | introns | | |
| J.000863F.000863F. 111241- | JL | | no | | flower |
| 112867 | | | introns | | |
| Group 4 -CsCBD | | PK-chr2, chr7 | | up to 6 | |
| | | PN-chr6 | | genes | |
| _R_PK.chr02 | PK | | no | | flower |
| 60319585 . . . 60321196 | | | introns | | |
| J.000692F.000692F.176431- | JL | | no | | flower |
| 178062 | | | introns | | |
| _R_PK.chr07 | PK | | no | | flower |
| 28773364 . . . 28774212 | | | introns | | |
| _R_PK.chr02 | PK | | no | | flower |
| 60412489 . . . 60414121 | | | introns | | |
| J.000692F.000692F.76354- | JL | | no | | flower |
| 77738 | | | introns | | |
| J.000692F.000692F.83902- | JL | | no | | flower |
| 84227 | | | introns | | |
| _R_CsCBDAS_AB292682 | | | no | | flower |
| | | | introns | | |
| _R_J.001055F.001055F.4195- | JL | | no | | flower |
| 5829 | | | introns | | |
| _R_FN.chr06 | FN | | no | | flower |
| 21837038 . . . 21838672 | | | introns | | |
| Group 7 | | | | | |
| _R_J.000493F.000493F.506409- | JL | PK-chr7 | no | up to 11 | |
| 507745 | | | introns | genes | |
| _R_J.001432F.001432F. 14412- | JL | | no | | flower |
| 15748 | | | introns | | |
| J.001682F.001682F.138453- | JL | PK-chr7 | no | | flower |
| 139524 | | | introns | | |
| PK.AGQN03004744.1 | PK | | no | | flower |
| 51696 . . . 53032 | | | introns | | |
| J.001008F.001008F.84862- | JL | | no | | |
| 85940 | | | introns | | |
| PK.chr07 | PK | | no | | flower |
| 52806167 . . . 52807512 | | | introns | | |
| PK.chr01 | PK | | no | | flower |
| 27733999 . . . 27735023 | | | introns | | |
| J.000161F.000161F.656278- | JL | PK-chr1 | no | | flower |
| 657204 | | | introns | | |
| J.000447F.000447F.501848- | JL | FN-chr3 | no | | flower |
| 503032 | | PK-chr3 | introns | | |
| _R_FN.chr03 | FN | | no | | flower |
| 10150821 . . . 10152435 | | | introns | | |
| FN.chr03 | FN | | no | | flower |
| 11785500 . . . 11792035 | | | introns | | |
| FN.chr03 | FN | | no | | flower |
| 10523782 . . . 10525193 | | | introns | | |
| _R_FN.QKVJ02002188.1 | FN | | no | | flower |
| 105614 . . . 112220 | | | introns | | |
| _R_PK.chr03 | PK | | no | | flower |
| 64666023 . . . 64667253 | | | introns | | |
| PK.AGQN03004229.1 | PK | | no | | flower |
| 65847 . . . 66514 | | | introns | | |
| PK.chr03 | PK | | no | | flower |
| 65145262 . . . 65146450 | | | introns | | |
| _R_J.000501F.000501F.437489- | JL | | no | | flower |
| 438840 | | | introns | | |
| J .000317F.000317F.716771- | JL | | no | | flower |
| 718196 | | | introns | | |
| _R_J.001363F.001363F.4383- | JL | | no | | flower |
| 5806 | | | introns | | |
| FN.QKVJ02000019.1 | FN | | no | | flower |
| 874669 . . . 876094 | | | introns | | |
| J.001111F.001111F.31566- | JL | PK-chr3 | no | | flower |
| 33200 | | | introns | | |
| PK.chr03 | FN | | no | | flower |
| 50895087 . . . 50896712 | | | introns | | |
| _R_FN.chr06 | FN | | no | | flower |
| 22244180 . . . 22245793 | | | introns | | |
| PK.AGQN03001586.1 | PK | | no | | flower |
| 35797 . . . 37401 | | | introns | | |
| J.000480F.000480F.148308- | JL | PN-chr6 | no | | flower |
| 149912 | | | introns | | |
| Group 1-CsCBCAS_THCAS like | | no chromozom | | 1 gene | flower |
| | | orientation, | | 5 copies | |
| | | PK chr07 | | | |
| PK.AGQN03010731.1 | PK | | no | | flower |
| 674 . . . 1830 | | | introns | | |
| PK.AGQN03006963.1 | PK | | no | | flower |
| 14285 . . . 15918 | | | introns | | |
| PK.AGQN03006292.1 | PK | | no | | flower |
| 7724 . . . 8880 | | | introns | | |
| FN.QKVJ02004136.1 | FN | | no | | flower |
| 3039 . . . 4672 | | | introns | | |
| PK.AGQN03005496.1 | PK | | no | | flower |
| 2986 . . . 4620 | | | introns | | |
| _R_CsCBCAS_THCAS like | | | | | |
| PK.AGQN03010271.1 | PK | | no | | flower |
| 2974 . . . 4605 | | | introns | | |
| J.001923F.001923F.49866- | JL | | no | | flower |
| 51502 | | | introns | | |
| FN.QKVJ02001794.1 | FN | | no | | flower |
| 69159 . . . 70796 | | | introns | | |
| J.000410F.000410F.385650- | JL | | no | | flower |
| 387287 | | | introns | | |
| _R_FN.QKVJ02004358.1 | FN | | no | | flower |
| 21738 . . . 23377 | | | introns | | |
| J.000410F.000410F.531453- | JL | | no | | flower |
| 533091 | | | introns | | |
| J.001832F.001832F.32483- | JL | | no | | flower |
| 34120 | | | introns | | |
| FN.QKVJ02001794.1 | FN | | no | | flower |
| 136709 . . . 138341 | | | introns | | |
| FN.QKVJ02004887.1 | FN | | no | | flower |
| 13940 . . . 15577 | | | introns | | |
| J.000410F.000410F.493426- | JL | PK-chr7 | no | | flower |
| 495063 | | | introns | | |
| Group 2- CsTHC | | | no | 1 gene | flower |
| | | | introns | | |
| _R_J.000692F.000692F.378942- | JL | | no | | flower |
| 380579 | | | introns | | |
| _R_CsTHCAS_AB212837 | | PK-chr7 | no | | flower |
| | | | introns | | |
| PK.chr07 | PK | | no | | flower |
| 28650050 . . . 28651687 | | | introns | | |
| Group 8 | | | no | 1 gene | flower |
| | | | introns | 1-2 copies | |
| PK.chr07 | PK | | no | | flower |
| 46549879 . . . 46551515 | | | introns | | |
| _R_FN.QKVJ02004488.1 | FN | | no | | flower |
| 6165 . . . 7801 | | | introns | | |
| FN.QKVJ02001794.1 | FN | | no | | flower |
| 9420 . . . 11056 | | | introns | | |
| J.000410F.000410F.436740- | JL | | no | | flower |
| 438377 | | | introns | | |
| J.001923F.001923F.108098- | JL | | no | | flower |
| 109729 | | | introns | | |

### Example 2

### Functional Analysis of Cannabinoid Genes by Gene Over Expression Using an Agrobacterium-Mediated Expression System in Cannabis sativa

A transgenic approach is used to determine the function of the uncovered genes in a cannabis callus culture, as exemplified on CBDAS and THCAS.

Materials and Methods

Plant Material

Shoots of C. sativa (cultivar #201) with 2-3 nodes were maintained under in vitro conditions on proliferation medium CRE (0.5 ppm m-Topolin, 1 MIS, 3% sucrose, 0.8% agar, pH 5.7). Leaves were excised, cut longitudinally and placed on regeneration medium CRF (0.2 ppm TDZ, 0.1 ppm NAA, 1 MIS, 3% sucrose, 0.8% agar, pH 5.7) for callus formation. After 1 month of incubation under a white fluorescent light (2000 lux) and 24±2° C. temperature with 40-50% relative humidity, generated calli were subjected to agrobacterium mediated transformation.

Agrobacterium Mediated Transformation

The Agrobacterium strain EHA105 harboring the desirable plasmid was grown in LB medium (10 g/l bacto-tryptone, 10 g/l NaCl, 5 g/l yeast extract) with antibiotics (50 μg/ml kanamycin) at 28° C. for 18-24 h. Then, agrobacterium was resuspended in a fresh transformation buffer (0.5 MS salts and full strength of B5 vitamins, 3% sucrose, pH 5.2 with 100 μM acetosyringone) to O.D 600=1, and further grown for 3 hours.

After 3 hours of incubation, the culture was transferred to a sterile vacuum chamber containing the callus. A pressure of 7 mBar was applied for 2 minutes under laminar air flow and the process was repeated 4 times. After vacuum infiltration process, the callus were transferred on CRF media containing 100 μM acetosyringone for 3 days. After co-cultivation the calli were treated with 200 ppm ticarticillin for 30-40 minutes followed by washes in sterilized double distilled water and drying. Following the treatment, the calli were transferred to CRF medium containing 200 ppm ticarticillin and kept under the same conditions as used for callus generation. After one week of incubation, half were transferred to metabolic analysis and half for further growth. Cultures were maintained on the same medium up to 2-3 sub-culture cycles.

GUS Assay

Detection of positive cells was carried out by an overnight incubation of callus cells in GUS buffer containing 0.1 M phosphate buffer, 100 ppm 5-Bromo-4-chloro-3-indolyl-beta-D-glucuronic Acid (X-gluc) and 20% methanol. The incubation was carried out at 37° C.

Plasmid Preparation

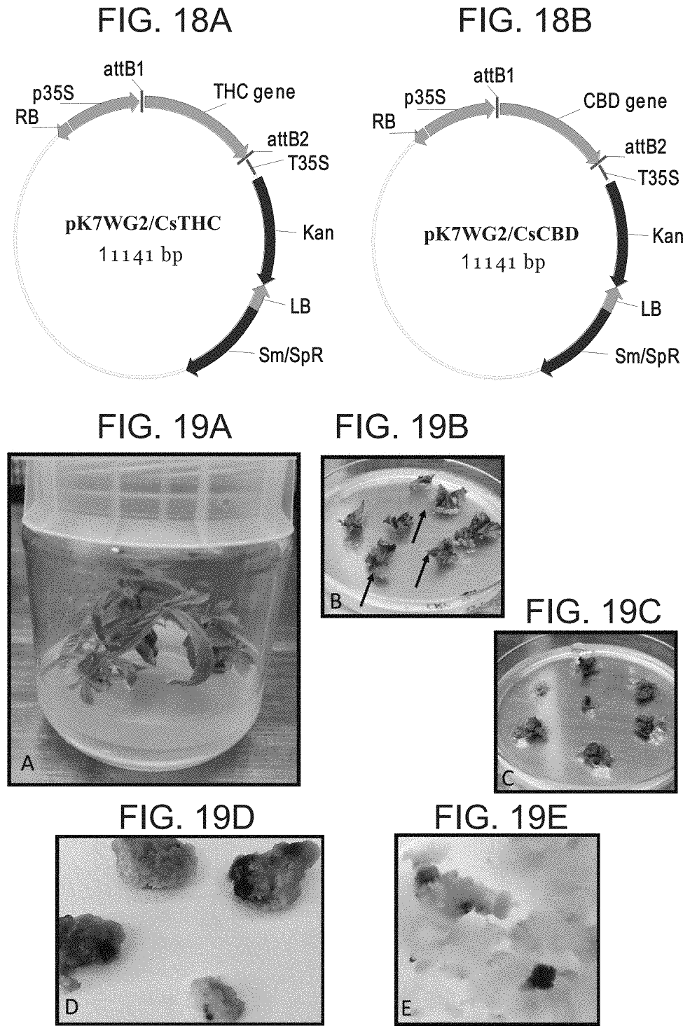

To clone CsTHCAS (SEQ ID NO: 173) and CsCBDAS (SEQ ID NO: 174) gDNA, Cannabis gDNA was isolated from young leaves approximately 2 wk old harvested in the morning by using C-TAB protocol. The CsTHCAS and CsCBDAS gDNA was amplified by using the primer set 5′-GGGGACAAGTTTGTACAAAAAAGCAGGCTATGAATTGCTCAGCATTTTCCTTTTGG-3′ (SEQ ID NO: 175) and 5′-GGGGACCACTTTGTACAAGAAAGCTGGGTATGATGATGCGGTGGAAGAGGTG-3′ (SEQ ID NO: 176) for CsTHC, and amplified by using the primer set 5′-GGGGACAAGTTTGTACAAAAAAGCAGGCTATGAAGTGCTCAACATTCTCCTT-3′ (SEQ ID NO: 177) and 5′-GGGGACCACTTTGTACAAGAAAGCTGGGTTAATGACGATGCCGTGGAAG-3′ (SEQ ID NO: 178) for CsCBD which was designed based on the sequence information of AB212837 for CsTHCAS and AB292682 for CSCBDAS and showed high sequence homologies. The amplified cDNA was cloned into the pDONR221 vector (Life Technologies) plasmid by a Gateway BP recombination reaction and the complete gDNA sequences were determined. CsTHC and CsCBD cDNA were transferred to the pK7WG2 plasmid (VIB) by a Gateway LR recombination reaction (Life Technologies) to make over expression plasmids: 35S:CsTHC and 35S:CsCBD binary vector (pK7WG2-CsTHC and pK7WG2-CsCBD) See FIGS. 18A-B.

HPLC Analysis

Sample Preparation from Tissue Culture for HPLC Analysis

1) Sample Preparation for Cali Samples

a) Cali samples were froze by liquid nitrogen, and dried by Lyophilization for three days until full dryness.

b) Samples were grinded with automated tissue homogenizer at 1200 RPM for 3.5 min.

c) 100 mg of each sample were weighed accurately into plastic tube, and 1 mL of Ethanol were added.

Sample Preparation for Leaves/Flowers/Shoots

a) Samples were dried at 40° C. overnight until full dryness.

b) Samples were grinded with automated tissue homogenizer at 1200 RPM for 3.5 min.

c) 100 mg of each sample were weighed accurately into plastic tube, and 4 mL of Ethanol were added.

2) Samples were vortexed for 30 sec, and shook mechanically for 20 min at 190 rpm.

3) Samples were filtered through 0.22 μm PTFE syringe filter into HPLC vial, and examined in HPLC.

4) The chromatographic conditions are based on Meiri et al., 2018. Separation was conducted using a Halo C18 column (3.0×150 mm, 2.7 μm) with a guard column (3.0×5 mm, 2.7 μm) (Advanced Material Technology, Wilmington, Del., USA) and a ternary A/B/C multistep gradient (solvent A: 0.1% formic acid in ULC/MS water, solvent B: 0.1% formic acid in acetonitrile, and solvent C: methanol, all solvents were of ULC/MS grade). Solvent C was kept constant at 5% throughout the run. The multistep gradient program was established as follows: initial conditions were 50% B raised to 67% B until 2 min, held at 67% B for 4 min, and then raised to 90% B until 10 min, held at 90% B until 14 min, decreased to 50% B over the next min, and held at 50% B until 20 min for re-equilibration of the system prior to the next injection. A flow rate of 0.5 mL/min was used, the column temperature was 35° C. and the injection volume was 1 μL.

Results

Over expression of the marker gene uidA (GUS) in callus cultures of C. sativa Agrobacterium strain EHA105 harboring plasmid pME504 with uidA gene for GUS expression were vacuum infiltrated to callus cultures #201. After 3 days co-cultivation calli were grown to allow further proliferation on regeneration medium. GUS staining was done 3 and 10 days after transformation (FIGS. 19A-E). The analysis after 3 days and 10 days indicates high transient GUS expression in the calli.

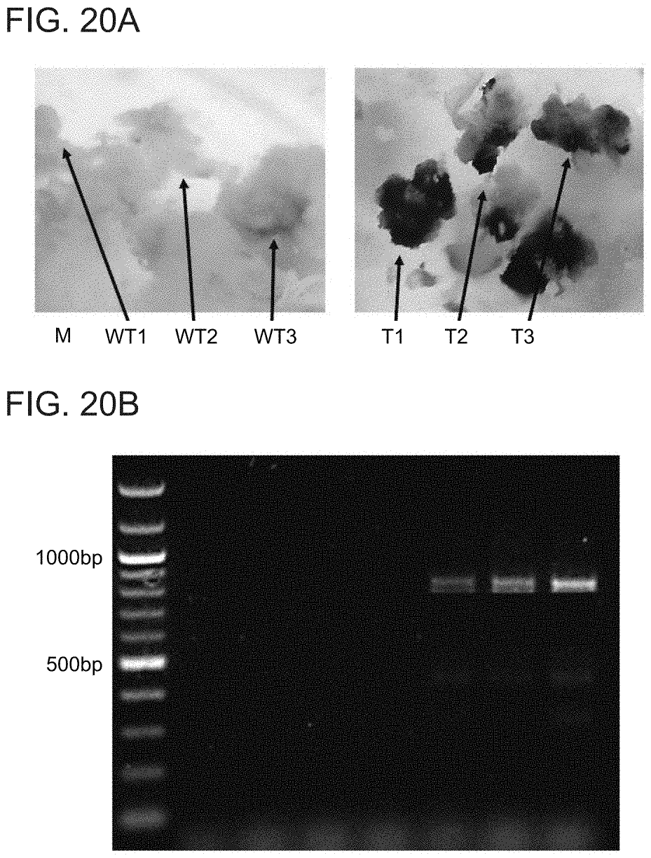

In order to get stable transformation, calli were growth for further proliferation on a proliferation medium containing selective antibiotic (Kan 100 mg/1). Calli were analyzed 30 days after transformation by GUS staining and PCR analysis (FIGS. 20A-B). The results indicate that some of the cells became transgenic cells with stable GUS over exasperation.

Over Expression of CBD in Callus Cultures of C. sativa

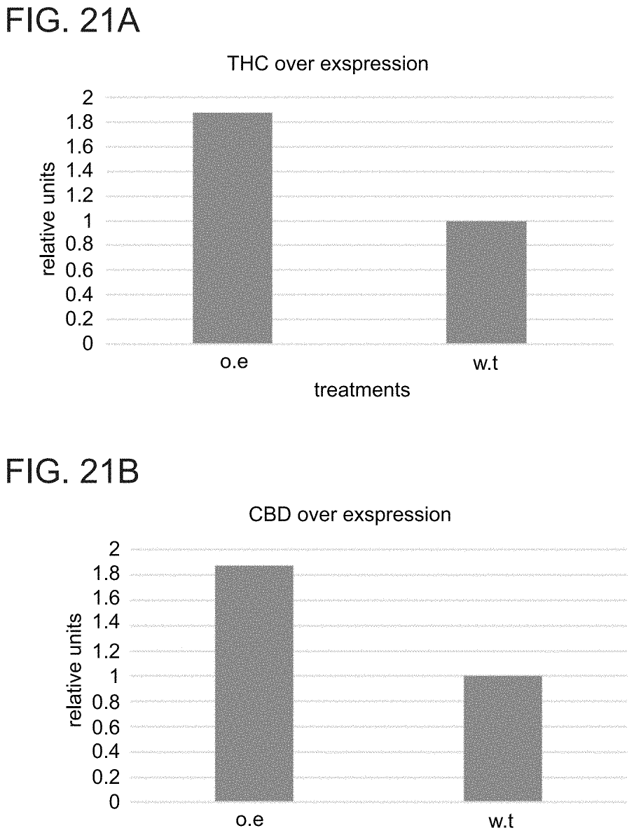

Agrobacterium strain EHA105 harboring plasmid pK7WG2-THC240 or pK7WG2-CBD157 were vacuum infiltrated to callus cultures #203. After 4 days co-cultivation callus were sampled for HPLC analysis (FIGS. 21A-B).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

Andre, C. M., Hausman, J. F., & Guerriero, G. (2016). Cannabis sativa: the plant of the thousand and one molecules. Frontiers in plant science, 7, 19.
ElSohly, M. & Gul, W. Constituents of Cannabis sativa in Handbook of Cannabis (ed. Pertwee, R.) 3-22 (Oxford University Press, New York, 2014).
Flores-Sanchez, I. J. & Verpoorte, R. (2008) Secondary metabolism in Cannabis. Phytochem. Rev. 7, 615-639.
Hanuš, L. O., Meyer, S. M., Muσoz, E., Taglialatela-Scafati, O. & Appendino, G. (2016). Phytocannabinoids: a unified critical inventory. Nat. Prod. Rep. 33, 1357-1392.
Kojoma, M., Seki, H., Yoshida, S., & Muranaka, T. (2006). DNA polymorphisms in the tetrahydrocannabinolic acid (THCA) synthase gene in “drug-type” and “fiber-type” Cannabis sativa L. Forensic Science International, 159(2-3), 132-140.
Kinghorn, A. D., Falk, H., Gibbons, S., & Kobayashi, J. I. (2017). Phytocannabinoids. Springer International Pu.
Laverty K U, Stout J M, Sullivan M J, Shah H, Gill N, Holbrook L, Deikus G, Sebra R, Hughes T R (2019) A physical and genetic map of Cannabis sativa identifies extensive rearrangements at the THC/CBD acid synthase loci. 29 (1):146-156.
Morales, P., Hurst, D. P., & Reggio, P. H. (2017). Molecular targets of the phytocannabinoids: a complex picture. In Phytocannabinoids (pp. 103-131). Springer, Cham.
Russo, E. B. & Taming, T. H. C. (2011). Potential cannabis synergy and phytocannabinoid-terpenoid entourage effects: Phytocannabinoid-terpenoid entourage effects. Br. J. Pharmacol. 163, 1344-1364.
Sirikantaramas, S., Morimoto, S., Shoyama, Y., Ishikawa, Y., Wada, Y., and Shoyama, Y. (2004). The gene controlling marijuana psychoactivity: molecular cloning and heterologous expression of Δ1-tetrahydrocannabinolic acid synthase from Cannabis sativa L. J. Biol. Chem. 279, 39767-39774
Taura, F., Dono, E., Sirikantaramas, S., Yoshimura, K., Shoyama, Y., and Morimoto, S. (2007b). Production of Delta(1)-tetrahydrocannabinolic acid by the biosynthetic enzyme secreted from transgenic Pichia pastoris. Biochem. Biophys. Res. Commun. 361, 675-680.
Turner, S. E., Williams, C. M., Iversen, L., & Whalley, B. J. (2017). Molecular pharmacology of phytocannabinoids. In Phytocannabinoids (pp. 61-101). Springer, Cham.
van Bakel, H., Stout, J. M., Cote, A. G., Tallon, C. M., Sharpe, A. G., and Hughes, T. R. (2011). The draft genome and transcriptome of Cannabis sativa. Genome Biol. 12:R102.
Volkow, N. D., Baler, R. D., Compton, W. M., & Weiss, S. R. (2014). Adverse health effects of marijuana use. New England Journal of Medicine, 370(23), 2219-2227.
Weiblen, G. D., Wenger, J. P., Craft, K. J., ElSohly, M. A., Mehmedic Z., Treiber, E. L., et al. (2015). Gene duplication and divergence affecting drug content in Cannabis sativa. New Phytol. 208, 1241-1250.

1. (canceled)

2. A method of producing cannabinoids in a plant, the method comprising modulating expression in the plant of at least one polypeptide comprising an amino acid sequence at least 95% identical to SEQ ID NO: 1-15 and 18-86 said polypeptide modulating cannabinoid synthesis, thereby producing cannabinoids in the cell.

3. A method of selecting a plant for a cannabinoid profile, the method comprising analyzing in the plant or part thereof presence of a nucleic acid sequence at least 95% identical to SEQ ID NO: 91-180 or amino acid sequence at least 95% identical to SEQ ID NO: 1-15 and 18-86, wherein presence or absence of said nucleic acid sequence or amino acid sequence is indicative of the cannabinoid profile.

4. The method of claim 3, further comprising determining a cannabinoid or cannabinoid profile of the plant or part thereof.

5. The method of claim 2, further comprising recovering the cannabinoids from the plant or cell.

6. The method of claim 5, wherein said recovering is by extraction and/or fractionation.

7-8. (canceled)

9. A cell, a plant, or part thereof having being genetically modified to down-regulate expression of a polypeptide comprising an amino acid sequence at least 95% identical to SEQ ID NO: 1-15 and 18-86.

10. The cell, plant or part thereof of claim 9, being a transgenic plant or plant cell.

11. The cell, plant or part thereof of claim 9, being a non-transgenic plant or plant cell.

12. The method of claim 3, wherein said modulating is by genome editing.

13. The method of claim 3, wherein said modulating is by transgenesis.

14. The method of claim 3, wherein said modulating is by breeding.

15. The method of claim 2, wherein said modulating comprises upregulating expression.

16. The method of claim 2, wherein said modulating comprises downregulating expression.

17. The cell of claim 9, wherein the cell is yeast.

18. (canceled)

19. The cell of claim 9, wherein the cell is a plant cell.

20. The cell or plant or part thereof of claim 9, wherein the plant part is a flower.

21. The cell or plant or part thereof of claim 9, wherein the plant part is a seed.

22. The cell or plant or part thereof of claim 9, wherein the plant part is a root.
